(19) United States
(12) Patent Application Publication
(10) Pub. No.: US 2006/0285697 A1
(43) Pub. Date: Dec. 21, 2006

(54) OPEN-AIR NOISE CANCELLATION FOR DIFFRACTION CONTROL APPLICATIONS

(75) Inventors: Masao Nishikawa, La Jolla, CA (US); Satoru Yukie, San Diego, CA (US)

Correspondence Address: INGRASSIA FISHER & LORENZ, P.C., 7150 E. CAMELBACK, STE. 325, SCOTTSDALE, AZ, 85251, US

(73) Assignee: COMFOZONE, INC., San Diego, CA (US)

(21) Appl. No.: 11/424,798

(22) Filed: Jun. 16, 2006

Related U.S. Application Data
(60) Provisional application No. 60/691,950, filed on Jun. 17, 2005.
(60) Provisional application No. 60/691,968, filed on Jun. 17, 2005.
(60) Provisional application No. 60/691,894, filed on Jun. 17, 2005.
(60) Provisional application No. 60/691,861, filed on Jun. 17, 2005.
(60) Provisional application No. 60/691,941, filed on Jun. 17, 2005.

(51) Int. Cl. A61F 11/06

(52) U.S. Cl. 381/71.1

(57) ABSTRACT

A variety of open-air noise cancellation systems are disclosed. The systems are configured to suit the needs of the particular application, for example, a sound wall installation, a seat or chair headrest application, a patio umbrella installation, or a window/door treatment application. A particular system may utilize an analog-based or a digital-based processing architecture that receives a noise signal, processes an out-of-phase noise cancellation signal, and generates an out-of-phase sound wave that effectively cancels the noise signal.

### CROSS-REFERENCE TO RELATED APPLICATION(S)

This application claims the benefit of U.S. provisional patent application Ser. No. 60/691,950, filed Jun. 17, 2005, U.S. provisional patent application Ser. No. 60/691,968, filed Jun. 17, 2005, U.S. provisional patent application Ser. No. 60/691,894, filed Jun. 17, 2005, U.S. provisional patent application Ser. No. 60/691,861, filed Jun. 17, 2005, and U.S. provisional patent application Ser. No. 60/691,941, filed Jun. 17, 2005. The contents of these provisional patent applications are incorporated by reference herein.

### TECHNICAL FIELD

The present invention relates generally to environmental noise control systems. More particularly, the present invention relates to an open-air noise cancellation system suitable for diffraction control.

### BACKGROUND

Environmental noise has become a very significant issue for many homes, businesses and other institutions. A variety of different factors contribute to the problem of environmental noise pollution. They include increasing population density, per capita space reduction, and increasing levels of industrial, transportation and residential noise.

Common noise sources include roads and freeways, airplanes, industrial institutions, plants and factories, air conditioners, pool equipment, and many others.

According to the United States Environmental Protection Agency and a host of other government and not-for-profit institutions, noise pollution is a significant environmental concern and may cause a variety of significant problems. For example, people exposed to transportation noise may experience such consequences as loss of sleep, productivity loss, hearing problems, loss of physical well-being, stress, and increasing health care costs.

Property values may also be lowered because of nearby transportation noise sources.

Accordingly, it is desirable to have systems, devices, and apparatus for reducing environmental noise. Furthermore, other desirable features and characteristics of the present invention will become apparent from the subsequent detailed description and the appended claims, taken in conjunction with the accompanying drawings and the foregoing technical field and background.

### BRIEF SUMMARY

A system is provided for reducing the effects of environmental noise by actively canceling noise which is diffracted over acoustic walls and other objects, which may be located between the noise source and the listener. The system reduces the amount of sound diffracted by the top edge of a wall, thus reducing the amount of environmental noise heard on the “protected” side of the wall. The example embodiment of the system has multiple microphones and speakers, which may be suitably aligned, paired, or unpaired. Each microphone provides accurate information on the characteristics of the noise elements and/or components, such as frequency, types, direction, and power. The noise information collected by the microphones is electronically processed to provide signals having the opposite phase of the noise signals. The out-of-phase signals are transferred to amplifiers for output to the speakers for generation of sound signals having the same magnitude but opposite phase of the noise, thus canceling the original noise signals.

### BRIEF DESCRIPTION OF THE DRAWINGS

A more complete understanding of the present invention may be derived by referring to the detailed description and claims when considered in conjunction with the following figures, wherein like reference numbers refer to similar elements throughout the figures.

FIG. 1 is a diagram of an environment separated from a noise source by a wall;

FIG. 2 is a diagram of an environment separated from a noise source by a wall capped with a sound absorbing material;

FIG. 3 is a perspective view of a top edge structure mounted to sound absorptive walls;

FIG. 4 is a schematic side view of a top edge structure for a wall;

FIG. 5 is a graph of Noise Reduction Coefficient (noise absorption ratio) by frequency and back space thickness for an aluminum porous material;

FIG. 6 is a perspective view of the inner frame of a top edge structure for a wall;

FIG. 7 is a schematic representation of an analog-based open-air noise cancellation system;

FIG. 8 is a schematic representation of a digital-based open-air noise cancellation system;

FIG. 9 is a schematic representation of an electronic gyro tracking system suitable for use with a noise cancellation system;

FIG. 10 is a schematic representation of gyro ear position sensors placed for horizontal movement tracking;

FIG. 11 is a side view of a noise cancellation system deployed in a headrest;

FIG. 12 is a schematic representation of a speaker suitable for use in a noise cancellation system;

FIG. 13 is a perspective view of a headrest with noise cancellation speakers;

FIG. 14 is a perspective view of a headrest with noise cancellation speakers and sound absorptive material;

FIG. 15 is a perspective view of a pop noise prevention feature for a headrest-mounted noise cancellation speaker;

FIG. 16 is a partially exploded perspective view of a headrest-mounted noise cancellation system;

FIG. 17 is a perspective view of foldable speakers suitable for use in a headrest noise cancellation system;

FIG. 18 is a schematic representation of a headrest noise cancellation system;

FIG. 19 is a perspective view of a headrest noise cancellation system implemented in a chair;

FIG. 20 is a schematic representation of an analog-based open-air noise cancellation system;

FIG. 21 is a schematic representation of a digital-based open-air noise cancellation system;

FIG. 22 is a perspective view of a noise cancellation system installed on a window shutter mechanism;

FIG. 23 is a diagram that represents the characteristics of a noise cancellation speaker;

FIG. 24 is a perspective view of a noise cancellation system installed on a window shutter mechanism;

FIG. 25 is a diagram that depicts a mechanical fin and blade assembly for a noise cancellation system;

FIG. 26 is a schematic representation of an analog-based noise cancellation system;

FIG. 27 is a schematic representation of a digital-based noise cancellation system;

FIG. 28 is a diagram of one example embodiment of a noise cancellation system for an umbrella implementation;

FIG. 29 is a diagram of another example embodiment of a noise cancellation system for an umbrella implementation;

FIG. 30 is a diagram of another environment having a noise cancellation system deployed therein;

FIG. 31 is a schematic representation of an analog-based noise cancellation system;

FIG. 32 is a schematic representation of a digital-based noise cancellation system;

FIG. 33 is a perspective view of a wall having noise cancellation speakers mounted thereon;

FIG. 34 is a perspective view of another wall having noise cancellation speakers mounted thereon;

FIG. 35 is a diagrammatic top view of a moving target detection and adjustment system; and

FIG. 36 is a front view of an example wall-mounted noise diffraction control system.

### DETAILED DESCRIPTION

The following detailed description is merely illustrative in nature and is not intended to limit the embodiments of the invention or the application and uses of such embodiments. Furthermore, there is no intention to be bound by any expressed or implied theory presented in the preceding technical field, background, brief summary or the following detailed description.

Embodiments of the invention may be described herein in terms of functional and/or logical block components and various processing steps. It should be appreciated that such block components may be realized by any number of hardware, software, and/or firmware components configured to perform the specified functions. For example, an embodiment of the invention may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments of the present invention may be practiced in conjunction with any number of environments in which noise cancellation or reduction may be desirable, and that the systems described herein are merely example embodiments of the invention.

For the sake of brevity, conventional techniques related to analog and digital signal processing, microphone and speaker design, acoustics, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in an embodiment of the invention.

The following description may refer to a “node” in the context of an electrical circuit or system. As used in this context, a “node” means any internal or external reference point, connection point, junction, signal line, conductive element, or the like, at which a given signal, logic level, voltage, data pattern, current, or quantity is present. Furthermore, two or more nodes may be realized by one physical element (and two or more signals can be multiplexed, modulated, or otherwise distinguished even though received or output at a common node).

The following description may refer to elements or nodes or features being “connected” or “coupled” together. As used herein, unless expressly stated otherwise, “connected” means that one element/node/feature is directly joined to (or directly communicates with) another element/node/feature, and not necessarily mechanically. Likewise, unless expressly stated otherwise, “coupled” means that one element/node/feature is directly or indirectly joined to (or directly or indirectly communicates with) another element/node/feature, and not necessarily mechanically.

Diffraction Control Apparatus

An example noise cancellation system as described herein may utilize a suitably configured apparatus that controls diffraction of sound waves from a wall. An apparatus is provided for reducing the effects of environmental noise by altering the diffraction behavior of sound waves. The apparatus reduces the amount of sound diffracted by the top edge of a wall, thus reducing the amount of environmental noise heard on the “protected” side of the wall. In one embodiment, the noise control apparatus includes a frame having a base and a contoured panel opposing the base, where the base is configured for coupling to a top edge of a wall and the contoured panel has a cross sectional shape that reduces diffraction of sound. The apparatus may also include an outer skin, formed from a sound absorbing material, surrounding at least the contoured panel. Thus, this particular apparatus leverages porous sound absorptive materials for open air noise reduction with certain top edge mechanical and acoustic optimization to control and reduce diffraction of sound, particularly in the outdoor environment.

In general, when there are noise issues, sound walls are installed between the source and the receiver of the noise. The height, location, and the materials of the sound walls play a significant role in determining the effectiveness of the sound walls. In general, the closer the wall is placed to the sound source, or to the receiver, the better the noise reduction effect. The higher the wall, the better the effect would be for noise reduction. However, there are many height limitations in installing walls, and making the walls too high reduces brightness of the space and increases psychological pressures.

Use of sound absorptive materials on the walls between the noise source and the receiver position can be generally effective in reducing noise and the amount of reflecting noise. Walls with reflective materials such as masonry and concrete create reflections of noise which may potentially increase the overall level of noise in the environment. Walls that are made of porous materials with many unequal holes and voids are considered to be absorptive materials.

There are two practical measurement criteria that can be used to determine the characteristics and effectiveness of the materials used for sound walls. Transmission Loss (or Sound Transmission Class=STC) is the sound energy transmitted through the wall from the sound source to the receiver when the wall is installed on the line of sight point. Usually, high Transmission Loss of approximately 30 dBA is considered to be associated with a good sound barrier. Absorption Ratio (or Noise Reduction Co-efficiency=NRC) is another criteria for determining how much of the sound energy is absorbed (and reflected) by such walls. For example, a wall with a 0.90 NRC rating means that 90% of the noise is absorbed, and 10% is reflected.

These criteria are good measurement criteria for the sound walls, however, there is another important path called the “diffraction path” where sound travels from the source to the receiver around an object. Diffraction is a physical phenomena where waves (whether light, sound, or water) travel around an object as though the waves bend around the object. In the case of a vertically deployed sound wall, sound may bend downward at the top of the wall, thus traveling towards the receive point of the sound. Other than direct sound paths, diffraction is one of the most significant paths of sound that can travel from the source to the receiver in an open air environment.

The diffraction amount depends on the length of the wave as well as the angles from the source through the object to the receiver. Sound with longer wavelength (lower frequency sound) diffracts more, and sound with shorter wavelength (higher frequency sound) diffracts less. The more angles from the source through the object to the receiver, the less sound diffracted. Audible sound frequencies are between 20 Hz to 20,000 Hz, which corresponds to wavelengths between 17 millimeters up to 17 meters. Sound travels at the speed of 340 meters per second, thus a frequency of 100 Hz corresponds to a 3.4 meter wavelength, and a frequency of 1,000 Hz corresponds to a 34 centimeter wavelength.

The understanding of sound, reflection, absorption, and diffraction has been increased in the recent years and many improvements in the sound walls have been implemented. However, traditional applications of absorptive materials have not addressed diffraction patterns for purposes of diffraction control to effectively reduce the unwanted noise.

An apparatus or system as described herein provides effective methods of reducing the diffraction of environmental noise. In the practical embodiment, the mechanics of a top edge structure (which can be installed on top of a wall) is covered with a porous absorptive material. In an ordinary wall without such a top edge structure, the sound starts to diffract when it reaches the top of the wall—the path of the sound forms an angle near the top of the wall and the diffracted sound travels downwards towards the receiver (e.g., a person). A top edge component configured in accordance with an example embodiment affects the sound in a different manner. In this regard, the sound diffracts at the mechanics of the top edge, however, it is absorbed by the porous material right next to the point of diffraction. The structure is physically wide enough to cover the frequencies (wavelength) in question, with a curved feature to further absorb the noise when sound intends to travel and diffract along the top edge of the wall. In addition, the structure has an overall length that can be selected to increase the angles of potential diffraction paths. The structure has a first circular edge, and a second circular edge to capture diffracted sound effectively (see FIG. 4).

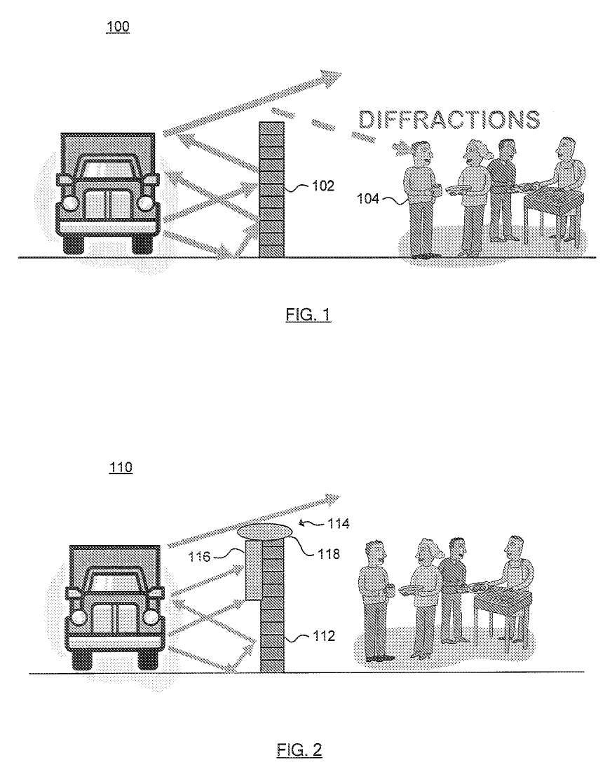

FIG. 1 is a diagram of an environment 100 separated from a noise source by a wall 102. FIG. 1 illustrates how, in normal environments, sound on one side of the wall 102 can travel over the wall 102, and how some of the sound can be diffracted downward such that it travels to a receiver 104 located on the other side of the wall 102.

In example embodiments, the sound diffracts when it reaches the top of the wall, however, it is also absorbed by the mechanism and the material of a top edge structure before the sound travels around the edge. FIG. 2 is a diagram of an environment 110 separated from a noise source by a wall 112 capped with a suitably configured apparatus 114. Apparatus 114 represents one practical implementation that provides a more effective way of reducing reflections using absorptive material on the noise side of the wall 112. In this example, apparatus 114 includes an absorptive material 116 mounted to the “noisy” side of the wall 112, and a top edge structure 118, which may be mounted to the top edge of the wall 112 and/or to the absorptive material 116.

The top edge structure eliminates or reduces diffracted noise because, when the noise signal hits the absorptive material instead of reflective material, some portion of the noise signal will be absorbed (where the absorbed component might otherwise be diffracted). In addition, as the diffracted noise travels over the top of the wall it also travels along the absorptive material, thus making the overall sound pressure level lower when it reaches the other end of the top edge structure. The sound pressure continues to spread out (lower frequencies being less directional, and higher frequencies being more directional), however, as the noise signal hits and travels over the top edge structure, the overall sound pressure level diminishes from the leading feature of the top edge structure to the trailing feature of the top edge structure.

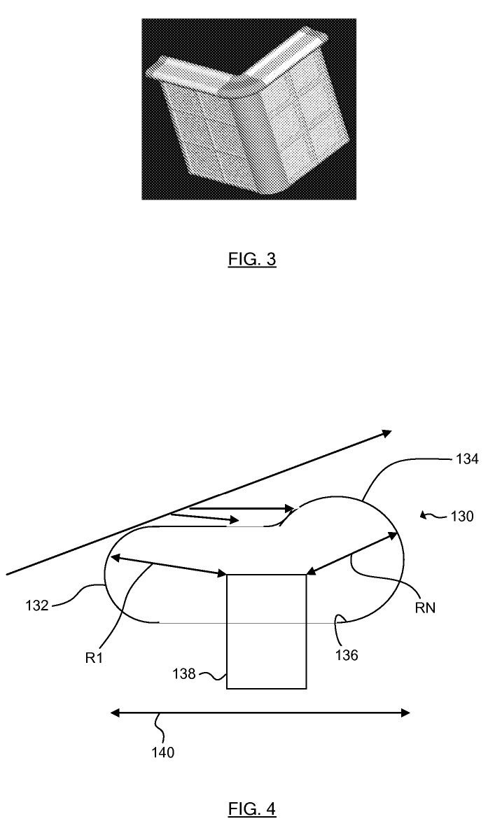

FIG. 3 is a perspective view of a top edge structure mounted to sound absorptive walls. FIG. 3 illustrates one example deployment of top edge structure 118 shown in FIG. 2. FIG. 3 shows how the top edge structure can be attached to sound absorptive walls in one example embodiment. As shown in FIG. 3, the top edge structure is contoured and shaped in a manner that enhances its performance. In this regard, FIG. 4 is a schematic side view of a top edge structure 130 for a wall. Top edge structure 130 is suitable for use in a deployment such as that depicted in FIG. 3. FIG. 4 shows how the first circular edge 132 diffracts the noise, but then the sound is absorbed by the absorptive materials and the structure. The second circular edge 134 captures the diffracted sound from the first circular edge 132, as well as diffracted noise by its own curve. The curvature or radius of the first edge 132 is less than the curvature or radius of the second edge 134. The structure's size, especially the width, is targeted to reduce a wide range of frequencies in the environmental noise characteristics, while, at the same time, having an aesthetically pleasant size to provide comfort behind the walls.

In a practical embodiment, the top edge structure 130 includes a frame 136 or skeleton that is covered with absorptive porous materials (not shown in FIG. 4). The frame 136 allows sound to be captured inside the structure. The interior of top edge structure 130 may be an open air buffer, or it may be filled with any suitable material. Top edge structure 130 may also include a rectangular, cylindrical, or other shaped back/bottom cover 138. Back/bottom cover 138 functions to stop the noise that has already been reduced in sound pressure level coming through the porous material from further going through the structure. Cover 138 also reflects such noise and cancels out the specific harmonics of resonance frequency tones that continuously enter and reflect by the back/bottom cover 138. In this example embodiment, the overall width (identified by arrow 140 in FIG. 4) of top edge structure 130 is at least 350 millimeters.

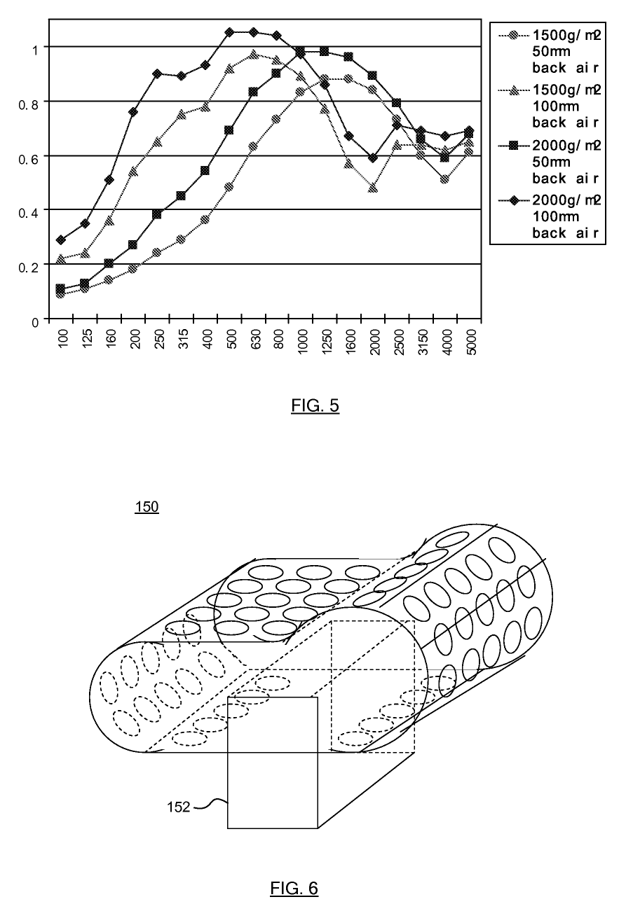

The absorptive material surrounding the frame 136 may be a full recyclable porous material made of aluminum fiber bonded to form a sheet by a continuous bonding process. Alternatively, the absorptive porous material may be made of fiberglass, if it is treated to reduce or prevent moisture absorption. An aluminum-based sheet is easy to cut and form to any shape and is therefore economical in applying to noise control products. Several examples of such a sheet has NRC characteristics as shown in FIG. 5. The vertical axis in FIG. 5 represents the NRC for the material, and the horizontal axis in FIG. 5 represents the frequency in Hz. FIG. 5 depicts the characteristics of sheets having different thicknesses (in grams per square meter) and back air buffer spacing (in millimeters). The back air buffer spacing is an open air space between the aluminum porous material and the back panel. It functions as a space to control the distance between the already-reduced noise coming into the structure versus the noise reflected by the back panel. This continuously creates an inside cancellation by resonance of frequencies. The more thickness, the more NCR, and the more back air buffer spacing (100 millimeters, for example) the more noise absorption in the lower central frequencies. In practice, moisture absorbed in the material can easily be dried (unlike glass fiber or other materials), so it is suitable for outdoor use.

FIG. 6 is a perspective view of an example inner frame 150 for top edge structure 130. In one embodiment, the inner frame 150 is made of plastic with many holes to support and hold the outer cover absorptive porous material. It also has an inner rectangular or cylindrical panel 152. Inside the frame 150 is an air buffer space. The buffer space and the distances from the surface of the porous material to the inner rectangular or cylindrical panels (R1 to RN in FIG. 4) can range from 50 mm to 100 mm so it covers wide range (or the target range) of central frequencies (between 200 Hz to 2500 Hz over NCR of 0.8 and more). This covers an adequate range of the characteristics of common environmental noises so that they can be reduced as they diffract. In general, the more air buffer space, the lower frequency noise is absorbed. The open holes account for approximately 50% of the overall surface area of the plastic frame 150 to let the noise travel through the aluminum porous material and the frame 150 into the air buffer space inside the frame 150 to form an absorptive characteristic.

Thus, an apparatus configured in accordance with an example embodiment leverages the combination of a porous absorptive material and a top edge structure to efficiently reduce unwanted environmental noise such as freeway noise by controlling the diffraction path over a sound barrier or wall. Systems, devices, and methods configured in accordance with example embodiments relate to:

A noise control apparatus comprising: a frame having a base and a contoured panel opposing the base, the base being configured for coupling to a top edge of a wall, the contoured panel having a cross sectional shape that reduces diffraction of sound; and an outer skin, formed from a sound absorbing material, surrounding at least the contoured panel.

A noise control system comprising: a wall structure for separating a noise source from a protected environment, the wall structure having a top edge; and a noise control apparatus coupled to the top edge, the noise control apparatus being configured to reduce diffraction of sound from the noise source into the protected environment. The noise control apparatus of the noise control system may comprise a frame having a base and a contoured panel opposing the base, the base being configured for coupling to the top edge, the contoured panel having a cross sectional shape that reduces diffraction of sound; and an outer skin, formed from a sound absorbing material, surrounding at least the contoured panel. The noise control system may further comprise a sound absorbing material coupled to the wall structure and facing the noise source.

Headrest Applications

An example noise cancellation system as described herein is suitable for use with headrests in seating applications. One example system is provided for reducing the effects of environmental noise by canceling noise in an open-air environment. The system provides effective open-air noise cancellation for headrest applications. The example embodiment of the system has two or more microphones and speakers, either paired or unpaired. Each microphone provides accurate information on the noise elements such as frequency, types, direction, and power of the environmental noises. Then the noise information from the microphones is electronically processed to provide sound having the opposite phase of the unwanted noise. The out-of-phase signals are transferred to amplifiers for output to the speakers for the same amount of sound simply in opposite phases to cancel the original noises. In this manner, the system uses active noise cancellation techniques in a headrest application suitable for use in an open-air environment, whether outdoor or indoor. The system is utilized to reduce background noise while people are seated, reclined, or laid down with the head rested.

Conventional active noise cancellation techniques leverage the so-called “closed air” and “feed back” environment. Such techniques are commonly used in headsets and cellular phones. In contrast, however, a system configured in accordance with the example embodiments described herein applies to the open-air environment, and such a system may employ one or more of the following techniques, features, and aspects (without limitation): active noise cancellation techniques; output power level control; frequency characteristic and control; mechanical and electronic/mechanical gyro tracking system; acoustic elements to control sound and noise; and open air optimization to offset open air noise.

The system may also combine the use of wireless audio functions, bass speakers, flat speakers, and/or pipe speakers. In one practical embodiment, the system is realized as a portable device which can be placed in any chair or seat, or incorporated in chairs such as for offices, living spaces, and airplane and automobile seats.

In one example embodiment, the system includes two or more microphones built in the speakers or physically separated from the speakers. The microphones detect the noise signals, change them to electrical signals for processing, and relay the processed signals to the speakers, which turn the signals back into sounds. The electronics create cancellation signals that are 180 degrees (within practical tolerances) out-of-phase with the actual noise signals. Thus, since the sounds from the speakers are of opposite phase from the noise, the generated sound actively cancels the unwanted noise sounds. The noise cancellation speakers add sound that is out of phase with the unwanted sound, and provides significant reduction of background noise.

A system as described herein provides effective methods and apparatus for implementing open-air noise cancellation for headrest applications. The sound from the speakers is out-of-phase with the noise, thus canceling the noise sound. The noise cancellation speakers reproduce loud noises that are simply out-of-phase, thus performing significant reduction of background noise and producing a very quiet environment for the listener.

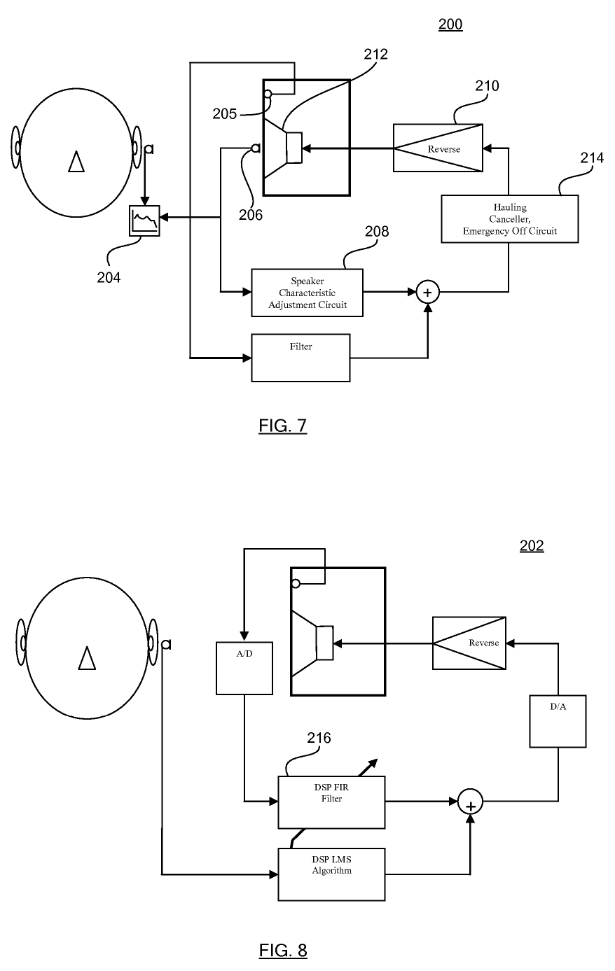

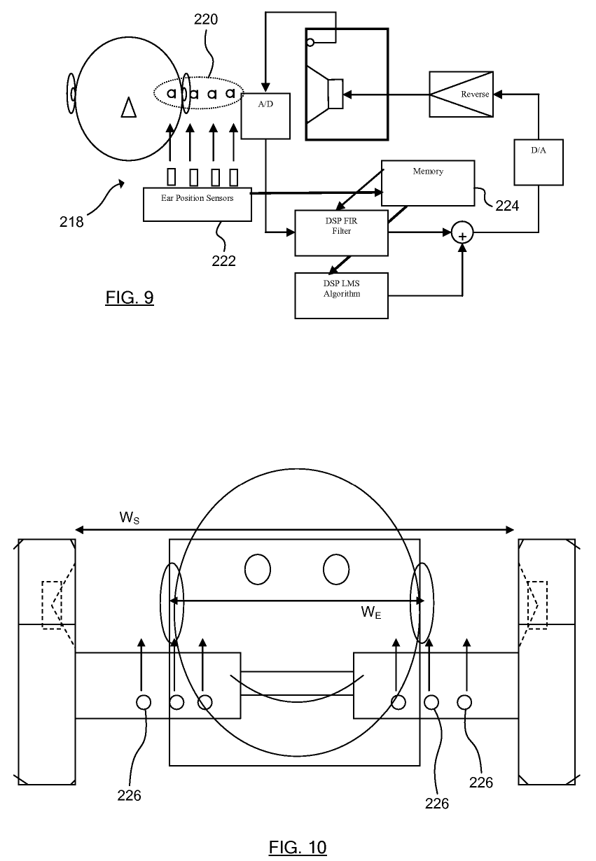

FIG. 7 is a schematic representation of an example analog-based open-air noise cancellation system 200 and FIG. 8 is a schematic representation of an example digital-based open-air noise cancellation system 202. In analog systems, in order to effectively cancel the noise in the target zone which is the zone close to the ear, it is important to measure the effect of sound travel (the distances from the noise collection microphone, the speaker, and the error microphone to the target zone), sound frequency characteristics created by the microphone specifications, speaker specifications, and other acoustic impacts such as from the speaker box, actual human head, and other objects. The sound characteristic acquisition system 204 essentially acquires such acoustic data at the time of development of the whole electronic and acoustic system. It measures the frequency characteristic and flatness of the sound system to properly reproduce opposite noise through the speakers. The system 200 may use a noise collection microphone 205 to obtain the unwanted noise signals. Once preset, the error correction microphone 206 collects the signals that are different from the preset signal characteristics, and adjustments are made in a speaker characteristic adjustment circuit 208. Then, a proper level of canceling noise dependent on such characteristic is reproduced and output through the reverse circuit 210 and the speaker 212. The hauling canceller and emergency shut of circuit 214 functions to reduce or shut off signals whenever there are excessive input to the microphones, that would otherwise create abrupt loud sound though the system.

In the digital system 202, similar development methods apply, however, in this system, analog signals are converted to digital data and processed digitally. The FIR filter 216 and DSP estimates the acoustic characteristics from the sensor to the error microphone and generates the signals which are reversed and output through the speaker for the target zone.

The following acronyms may be used herein, particularly with reference to FIGS. 7-10 and 18:

A/D: analog to digital signal converter;

D/A: digital to analog signal converter;

DSP: digital signal processing/processor, which may be used to digitally process the signals;

FIR filter: finite impulse response filter, which may be used to estimate the acoustic response from the sensor to the error microphone;

IR: infra red, which may be used in devices for detecting objects;

LMS Algorithm: Least Mean Squares Algorithm, which may be used to generate error signals;

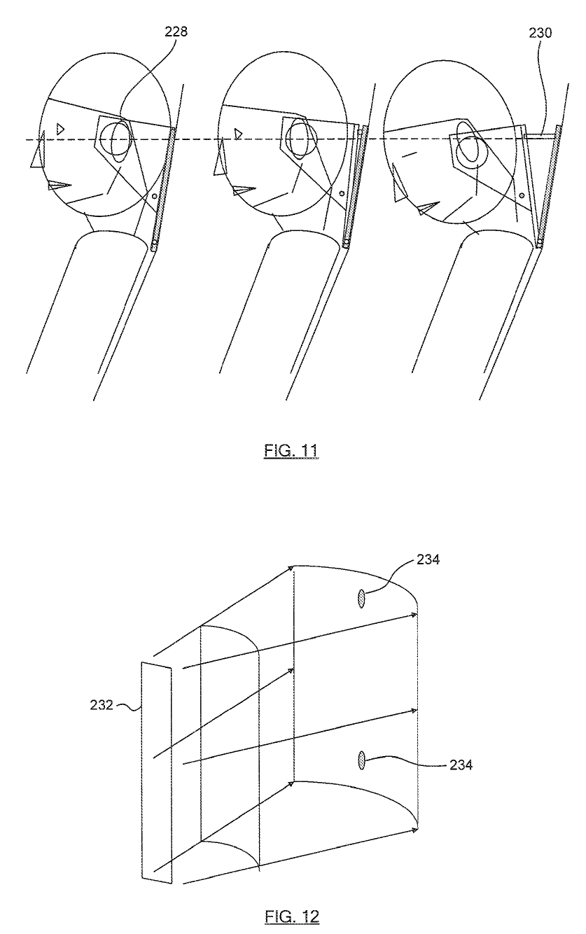

FIG. 9 is a schematic representation of an electronic gyro tracking system 218 suitable for use with an example noise cancellation system. The electronic gyro tracking system first calculates and records to a memory chip at the development stage separately the optimum power output levels, frequency characteristics of the microphones and the speakers, and the sound travel delays, based on the distance and location between the ear and the speaker (E1 to EN) to effectively provide 180 degrees opposite sound. These different distances correspond to the different microphone positions in the dashed oval 220 of FIG. 9. Then, after the development, the sound frequency characteristics acquisition system or the error correction microphone will be removed from the ear location. By one or more separate ear location detection sensors 222, either with IR (Infrared), laser, any other wireless technique, and image capturing devices, or any related detectors, the memory 224 puts out an optimum output level, the sound characteristics, and the delays to best cancel the noise without the microphones placed at the ear position. In practice, memory 224 stores data corresponding to the different distances E1 to EN for use in the tracking.

FIG. 10 is a schematic representation of gyro ear position sensors placed for horizontal movement tracking. FIG. 10 shows one example physical implementation of gyro ear position sensors 226, which may be used in the tracking system 218 described above. This example uses six IR sensors; three for each side of the user's head. In this model, the distance between the headrest speakers (corresponding to the dimension W_{S} in FIG. 10) is about 40-45 centimeters, and the distance between the user's ears (corresponding to the dimension W_{E} in FIG. 10) is about 22-25 centimeters. This arrangement enables effective active noise cancellation of an open-air environment without anything touching the ears. The concept also applies to vertical movements and to forward and back movements.

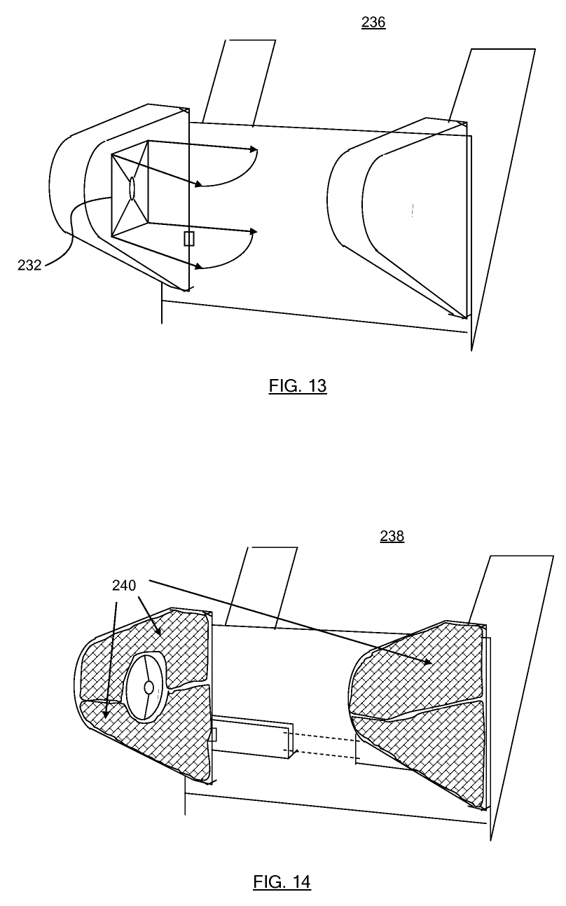

FIG. 11 is a side view of a noise cancellation system deployed in a headrest. FIG. 11 shows an example embodiment that utilizes a mechanical gyro tracking system for the headrest application. The speakers 228 that reproduce 180 degrees opposite noise for canceling the noise around the ear move forward and back with the motion of the head and the ear. In this example, the speakers 228 are attached to an accessory coupled to the chair and a spring element 230 is used to push forward as the head moves forward so that the speakers 228 follow the front and back motion of the ears.

FIG. 12 is a schematic representation of a speaker suitable for use in a noise cancellation system. FIG. 12 shows the characteristics of a panel, pipe, or flat speaker 232, and the use of such speakers for a tracking system in an open-air noise cancellation system for headrest applications. Since the sound produced by the panel, pipe, or flat speaker 232 travels as in the surface shape of a part of a cylinder, not as in the surface shape of a part of a sphere, there is no delay in sound travel between different vertical ear positions (E1-EN). Two possible vertical ear positions are identified by the small ovals 234 in FIG. 12. Thus, this sound pattern can provide on-time arrival of the canceling noise regardless of the vertical ear position relative to the speaker 232. In practice, at least one speaker 232 is used for each side of the head, and more than two speakers 232 can be used on each side or location for a multi-channel system.

FIG. 13 is a perspective view of an example headrest 236 with noise cancellation speakers incorporated therein. FIG. 13 shows one practical implementation of the panel, pipe, or flat speaker 232 for the tracking system. In practice, one speaker 232 is used for each side of the headrest 236.

FIG. 14 is a perspective view of a headrest 238 with noise cancellation speakers and sound absorptive material 240. FIG. 14 shows an example application of acoustic sound absorptive materials 240 surrounding the speaker boxes of the headrest noise cancellation system. Suitable sound absorptive materials 240 for use in this application are, without limitation: aluminum fiber porous material with some back air spacing, and other porous materials. The acoustic absorptive materials 240 surrounding the speaker box reduces diffraction and absorbs especially higher frequency unwanted noise coming towards the near ear zone.

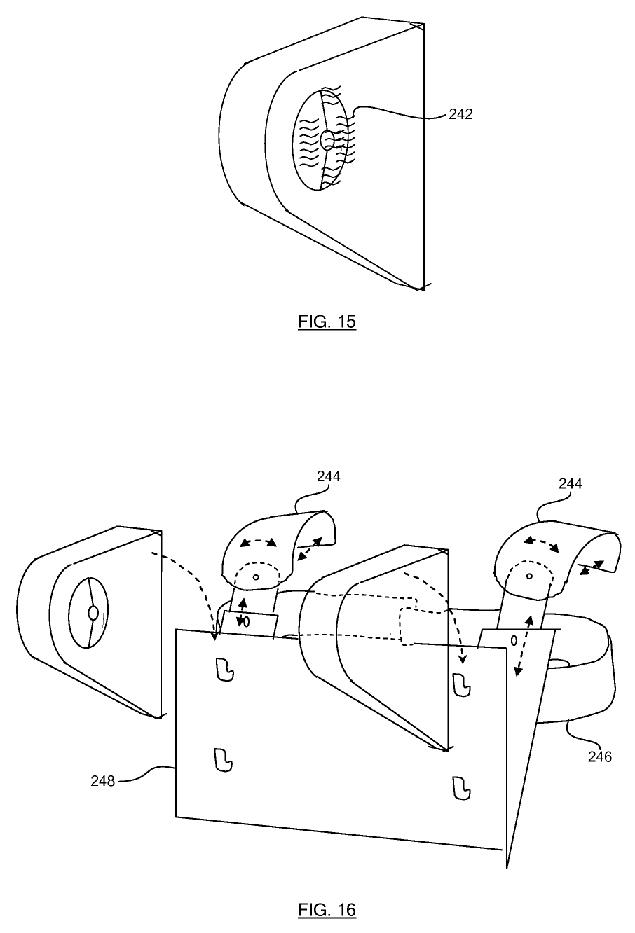

FIG. 15 is a perspective view of a pop noise prevention feature for a headrest-mounted noise cancellation speaker. FIG. 15 shows an example embodiment of a pop noise prevention feature 242 attached to the microphone of the headrest noise cancellation system. This feature prevents wind pop noise when gathering noise information by the microphone placed close to the speaker. In addition to the conventional pop noise reduction method using a mesh cover made of thin steel or aluminum strings, applied over the top of the microphones whether single, double, or triple mesh, a number of hair-like soft, but yet standing firm fur are placed from the conventional cover of the microphone. Such fur reduces the wind force without creating additional noise by the wind, lets through the sound or noise, and prevents the microphone receiver structure from creating so-called pop noise. For example, thin hair, wool, fur, pile, or the like are placed in front of the microphone cover to prevent capturing pop wind noise.

FIG. 16 is a partially exploded perspective view of an example headrest-mounted noise cancellation system. FIG. 16 shows an example embodiment, which can be attached to any size of existing chairs or seats with adjustable arms 244. Each arm can be a flexibly widening arm that can be adjusted in the forward and back direction as depicted by the arrows in FIG. 16. Each arm can also rotate about a pivoting mounting point, as depicted by the curved arrows. Each arm may also be adjustable or expandable in height, as depicted by the vertical adjustment arrows. The assembly may include mounting straps 246 for securing the assembly to the chair or headrest. Mounting straps 246 may be formed from hook-and-loop fastener material (e.g., VELCRO material), or the like. The flexible widening arms 244 can be made of, as an example, spring stainless steel covered by aesthetic leather cover. The arms 244 can widen by external force, but narrow back by their own force, thus grabbing and holding the shoulder of a subject chair upon which the system is placed. The rotating feature of the arms 244 is designed to fit any slope of each side of the shoulders of a chair so that the arms 244 can grab the shoulder effectively. The vertical adjustment feature allows arms 244 to fit a variety of heights of the shoulders of the chairs, so that the height of the speakers of the system that are hooked to a base plate can be adjusted to the height of the ears when a person is seated. The base 248 of the structure (to which the speakers are mounted using, for example, L-type or H-type hooks) is a plate which is connected to a belt that has rugged fasteners (like the Velcro brand loop fasteners) on the ends so that the belt can be easily and firmly tied, and held to the chair or the seat.

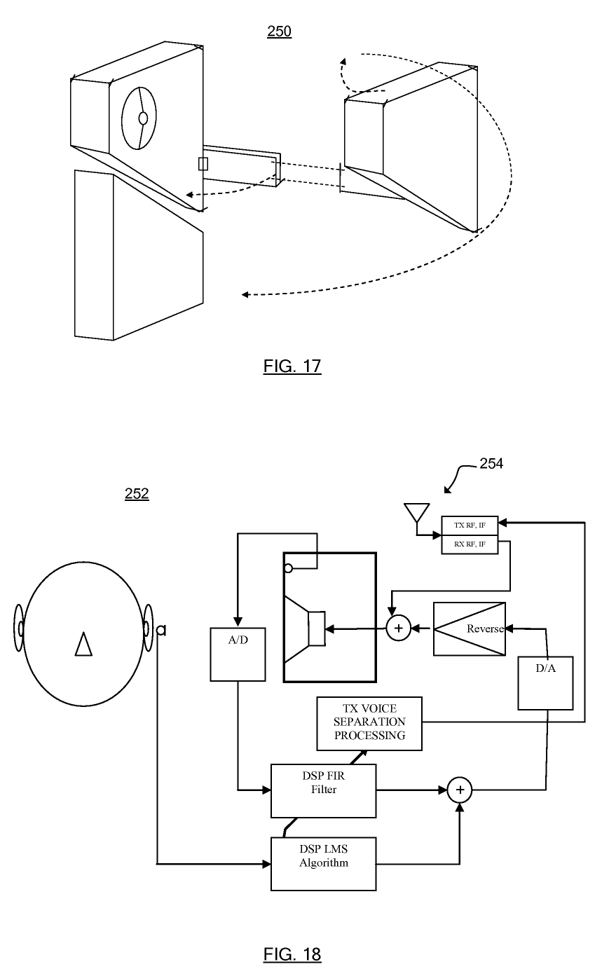

FIG. 17 is a perspective view of foldable speakers 250 suitable for use in a headrest noise cancellation system. In this example embodiment, the speakers fold 90 degrees to the x-axis, then one of the speakers rotates 180 degrees to match or mate with the other speaker. In this manner, the speakers fold into a size of a book or a note book personal computer. The dashed lines and arrows in FIG. 17 illustrate this folding technique.

FIG. 18 is a schematic representation of another example headrest noise cancellation system 252. This block diagram depicts a wireless or wired input function for the input of additional audio signals to the headrest noise cancellation system 252. The wireless or wired audio connection is achieved using any suitable wireless data communication technology, such as IEEE 802.11, IEEE 802.16, BLUETOOTH wireless technology, or other wireless or wired standards or non-standard technologies. The wireless port 254 allows the input of external audio signals such as, but not limited to music, radio, wireless or wired phone conversation, and environmental audio programs. FIG. 18 also shows an application for utilizing the noise detection microphones to be used in capturing voice conversation, which is separated from the noise information digitally processed, and is transmitted from the wireless connection platforms from the headrest open-air noise cancellation system. Thus, the headrest open-air noise cancellation system enables any additional audio input and output signals to be presented either through a wireless platform or a wired platform.

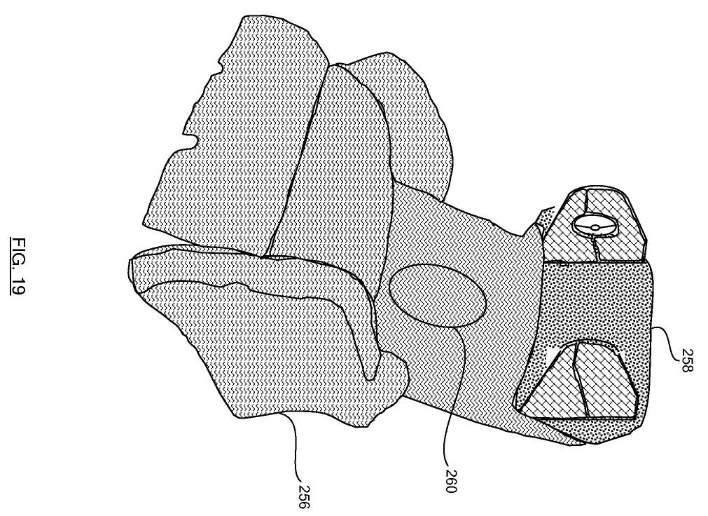

FIG. 19 is a perspective view of a headrest noise cancellation system implemented in a chair 256 or a seat. In the example shown in FIG. 19, the system is incorporated in the headrest 258 of the chair 256, which may be suitable for offices, living room sofas, airplanes, automobiles, beds, etc. FIG. 19 also shows bass, sub woofer, or sub bass speakers 260 connected to the system and mounted inside the chair 256. Since lower frequency sound does not have directional sound travel characteristics, the bass, sub woofer, or sub bass speakers 260 may be placed in any location close to the system, including beneath the seating positions or inside the backrest of the chair 256.

The systems described herein allow cancellation and reduction of background noise such as the highway traffic noise, the airplane noise, industrial noise, air conditioner and home equipment noise, office noise, and other noise in the open-air environment, as a portable device or as an installed device. Systems, devices, and methods configured in accordance with example embodiments relate to:

A noise cancellation system for open-air applications, the system comprising: open-air speakers configured for mounting in a listening position proximate to a listener's ears; a noise collection microphone located proximate to the open-air speakers, the noise-collection microphone being configured to obtain a noise signal; and a processor configured to generate a noise cancellation signal based upon the noise signal. The system may further comprise a tracking system configured to determine positioning of the listener's ears, wherein the processor is configured to generate the noise cancellation signal in response to the positioning of the listener's ears. The system may further comprise a forward biasing headrest device coupled to the open-air speakers, the forward biasing headrest device being configured to maintain a position of the open-air speakers relative to the listener's ears in response to forward movement of the listener's head. The open-air speakers may be configured to generate sound having a substantially cylindrical radiation pattern. The system may further comprise acoustic sound absorbing material located proximate to the open-air speakers. The system may further comprise wind noise reduction material located proximate to the open-air speakers. The system may further comprise means for combining an audio input signal with the noise cancellation signal.

A noise cancellation system for open-air applications, the system comprising: a seating structure having a headrest; open-air speakers mounted on the headrest; a noise collection microphone located proximate to the open-air speakers, the noise-collection microphone being configured to obtain an open-air noise signal; a processor configured to generate a noise cancellation signal that is directly out-of-phase with the noise signal; and a driver arrangement coupled to the open-air speakers, the driver arrangement being configured to drive the open-air speakers with the noise cancellation signal.

Window or Door Applications

An example noise cancellation system as described herein is suitable for use with an open window or door. A system is provided for reducing the effects of environmental noise by canceling noise in an open-air environment. The system provides effective open-air noise cancellation for open window or door applications. The example embodiment of the system has multiple microphones and speakers aligned, either paired or unpaired. Each microphone provides accurate information on the noise elements such as frequency, types, direction, and power of the environmental noise. Then, the noise information from the microphones is electronically processed to provide a signal or signals having the opposite phase of the noise signals. The out-of-phase signals are transferred to amplifiers for output to the speakers for the same amount of sound in opposite phases to cancel the original noises.

The active noise cancellation techniques described herein can be deployed in an open window or door application suitable for use in an open-air environment. The system is utilized to reduce noise coming in from the open window or door while people are inside on the other side of the noise source.

Conventional active noise cancellation techniques leverage the so-called “closed air” and “feed back” environment. Such techniques are commonly used in headsets and cellular phones. In contrast, however, a system configured as described below applies to the open-air environment, and such a system may employ one or more of the following techniques, features, and aspects (without limitation): active noise cancellation techniques; output power level control; frequency characteristic and control; acoustic elements to control sound and noise; and open air optimization to offset open air noise.

The system may also combine the use of flat and/or pipe speakers. In one practical embodiment, the system is deployed in conjunction with window shutters, and the system is suitably configured to reduce noise that might otherwise pass through the open window (or even through the glass of a closed window).

In one example embodiment, the system includes multiple sets of microphones and speakers. The microphones detect the noise, and the system processes the detected noise signals to create compensating or canceling electrical signals. The canceling signals are relayed to the speakers, which turn the generated signals back into sounds. The electronics create cancellation signals that are 180 degrees (within practical tolerances) out-of-phase with the detected noise signals. Thus, since the sounds from the speakers are of opposite phase from the noises, the generated sound actively cancels the unwanted noise sounds. The noise cancellation speaker(s) add counter-noise that is out of phase with the detected noise signals, and provide significant reduction of background noise.

One practical embodiment provides effective methods and apparatus for implementing open-air noise cancellation for open window or door applications. The sound from the speakers is out-of-phase with the noise, thus canceling the noise sound. The noise cancellation speakers reproduce loud noises that are simply out-of-phase, thus performing significant reduction of background noise and producing a very quiet environment.

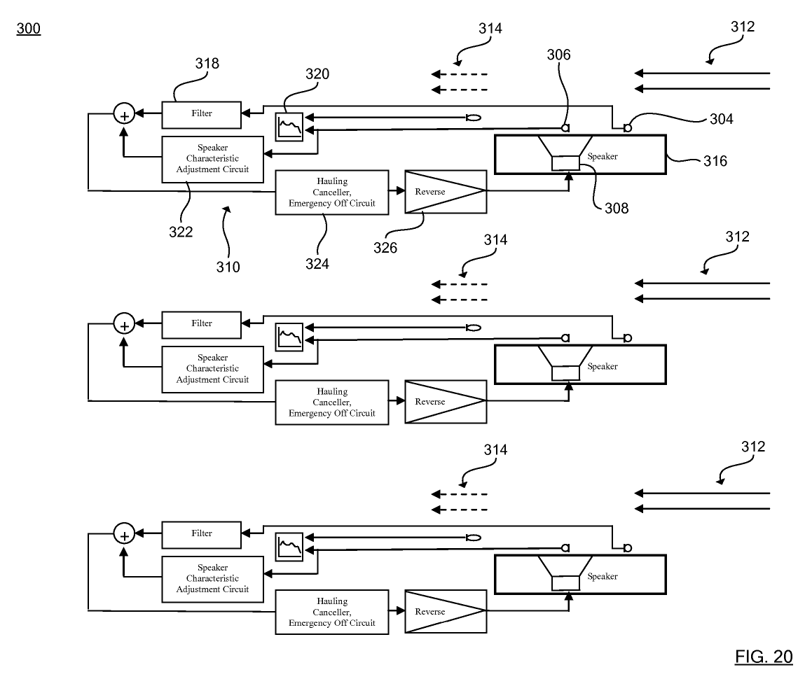

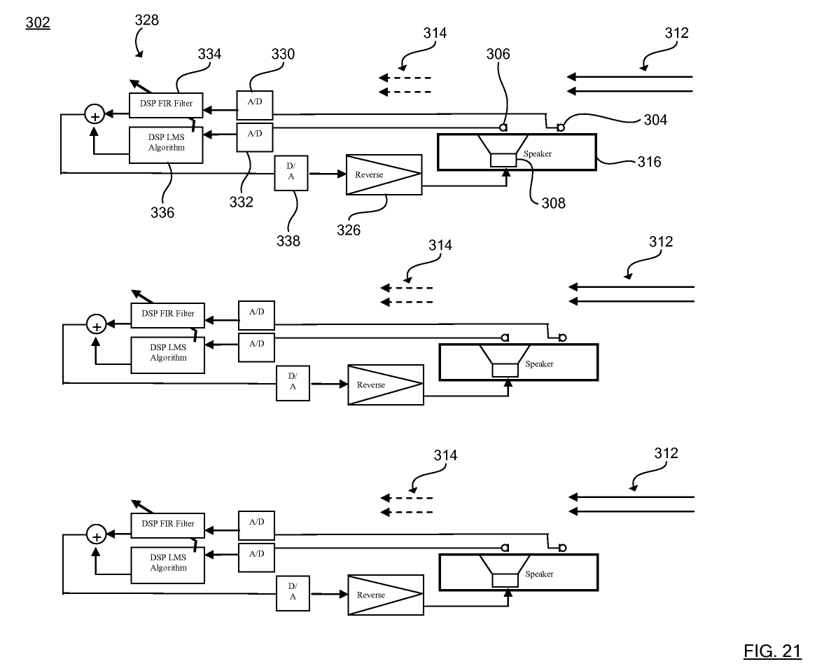

FIG. 20 is a schematic representation of an analog-based open-air noise cancellation system 300, and FIG. 21 is a schematic representation of a digital-based open-air noise cancellation system 302. These systems represent two possible practical implementations of a window/door application. In both the analog system 300 and the digital system 302, lines of speakers and microphones are aligned. FIG. 20 and FIG. 21 depict only three lines of speakers and microphones; in practice, however, any number of lines may be utilized. Each line may represent a noise cancellation subsystem for the respective system.

Each subsystem of analog system 300 generally includes at least one noise collection microphone 304, at least one error correction microphone 306, at least one speaker 308, and processing components 310 that are suitably configured to perform the noise cancellation techniques described herein. In particular, analog system 300 processes received noise 312, and generates cancelling signals at speaker 308, resulting in reduced noise 314 experienced by the listener.

Speaker 308 may be surrounded by a suitable enclosure or box 316. In practice, the box 316 may be realized as a shutter frame, and one shutter frame may serve as the box 316 for more than one speaker 308. Microphone 304 detects the noise 312 approaching the shutter frames, then the detected signals are processed by processing components 310, which may perform error correction and reverse the detected signals by 180 degrees for reproduction from speaker 308. Processing components 310 may include, without limitation: a filter 318; a sound characteristics acquisition subsystem 320; a speaker characteristic adjustment circuit 322; a hauling canceller and emergency off circuit 324, and a reverse circuit 326.

Use of such an active sound canceling array significantly reduces the overall noise going through the shutters. In analog system 300, in order to effectively cancel the noise in the target zone (e.g., the area on the interior side of the window), it is important to measure the effect of sound travel (the distances from the noise collection microphone 304, the speaker 308, and the error correction microphone 306 to the target zone), sound frequency characteristics created by the microphone specifications, speaker specifications, and other acoustic impacts such as the form of the speaker box 316 and other objects. The sound characteristics acquisition subsystem 320 essentially acquires such acoustic data at the time of development of the entire electronic and acoustic system. This subsystem 320 measures the frequency characteristic and flatness of the sound system to properly reproduce opposite noise through the speakers. Once preset, the error correction microphone 306 collects the signals that are different from the preset signal characteristics, for purposes of adjustment by the speaker characteristic adjustment circuit 322. Then, a proper level of canceling noise dependent on such characteristics is reproduced and output through the reverse circuit 326 and the speaker 308. The hauling canceller and emergency off circuit 324 functions to reduce or shut off signals whenever there are excessive inputs to the microphones, which would otherwise create abrupt loud sound though the system 300.

Each of the three subsystems shown in FIG. 20 may be identical in configuration and functionality. Thus, the sound characteristics acquisition subsystem 320, the speaker characteristic adjustment circuit 322, and other processing components 310 in each subsystem may function in response to the operation of one or more other subsystems. In other words, the three subsystems shown in FIG. 20 preferably cooperate with one another for purposes of overall noise cancellation.

In digital system 302, a similar technique applies, however, in digital system 302, analog signals are converted to digital data and for digital processing by processing logic 328. Digital system 302 may share several components and respective functionality with analog system 300; common features and functionality will not be redundantly described in the context of digital system 302.

Each subsystem of digital system 302 generally includes at least one noise collection microphone 304, at least one error correction microphone 306, at least one speaker 308 enclosed by a box 316, and processing logic 328 that is suitably configured to perform the noise cancellation techniques described herein. In particular, digital system 302 processes received noise 312, and generates cancelling signals at speaker 308, resulting in reduced noise 314 experienced by the listener.

Microphone 304 detects the noise 312 approaching the shutter frames, then the detected signals are processed by the digital processing logic 328, which may perform error correction and reverse the detected signals by 180 degrees for reproduction from speaker 308. Processing logic 328 may include, without limitation: analog-to-digital converters 330/332; an FIR filter 334; an LMS algorithm module 336; and a digital-to-analog converter 338. Digital-to-analog converter 338 is coupled to a reverse circuit 326.

The FIR filter 334 and its associated digital signal processor (DSP) estimates the acoustic characteristics from the sensor to the error microphone and generates signals which are reversed and output through the speaker 308 for the target zone.

Each of the three subsystems shown in FIG. 21 may be identical in configuration and functionality. Thus, the digital processing logic 328 and other components in each subsystem may function in response to the operation of one or more other subsystems. In other words, the three subsystems shown in FIG. 21 preferably cooperate with one another for purposes of overall noise cancellation.

In FIG. 21, the following acronyms may be used:

A/D: analog to digital signal converter;

D/A: digital to analog signal converter;

DSP: digital signal processing, which may be used to digitally process the signals;

FIR filter: finite impulse response filter, which may be used to estimate the acoustic response from the sensor to the error microphone; and

LMS Algorithm: Least Mean Squares Algorithm, which may be used to generate error signals.

The number of shutter frames, the number of microphones, and the number of speakers may depend on the height and the width of the target window or door. The spacing intervals between the speakers are desired to be less than 250 cm to effectively cancel open air noise in this application. The closer the speakers the better to cancel noise in higher frequency ranges. For example, 250 cm is equal to approximately half wavelength of a 680 Hz signal, thus making the noise cancellation effective below that frequency.

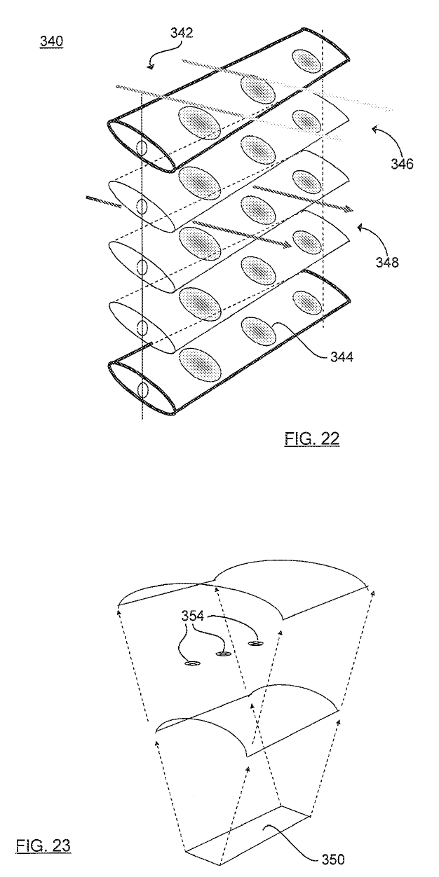

FIG. 22 is a perspective view of a noise cancellation system installed on a window shutter mechanism 340. The shutter frames, which may also function as speaker boxes for the window or door noise cancellation system, may be surrounded by acoustic sound absorptive materials. The sound absorptive material may be, for example, aluminum fiber porous material with some back air spacing, and other porous materials. The acoustic absorptive materials surrounding the speaker box reduces diffraction and absorbs especially higher frequency unwanted noise coming towards and going through the shutter frame. The frame maybe rectangular, oval, cylindrical, or any suitable shape to best absorb the noise from the acoustic perspective. As the noise 342 approaches the shutter mechanism 340, the electric noise sensor microphone detects the noise sound, the system processes the signals as described above, and outputs reverse phase noise from the speakers 344, thus actively canceling the noise at that point. Reduced or cancelled noise 346 is experienced by listeners on the other side of shutter mechanism 340. In this embodiment, multiple sets of the microphones (not separately shown) and the speakers are aligned along the frame of each shutter. The shutters can be rotated to have visual shutter effect, yet the noise cancellation continues to be performed with the electronic error correction feed back. When open, the shutters allow fresh air 348 to pass through the window/door.

The electronics components (such as DSPs, A/D converters, and D/A converters) can be located inside the frames or external to the frames in a box which may be mounted on the wall. The electronics can be connected to the microphones and the speakers 344 facing the surface of the frames by wires. AC power can be converted to DC power and supplied to the electronics mounted on the wall or inside the frames.

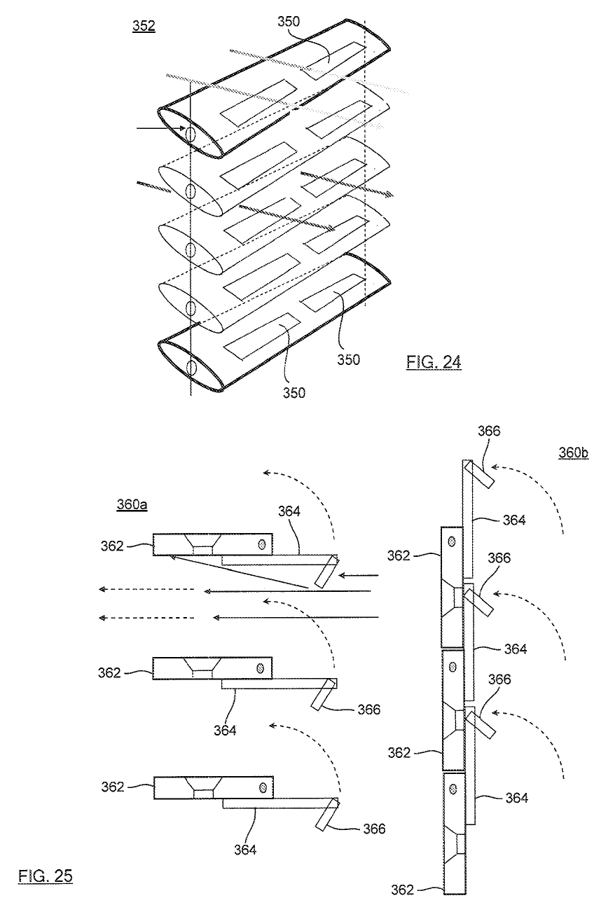

FIG. 23 is a diagram that represents the characteristics of a noise cancellation speaker 350, and FIG. 24 is a perspective view of a noise cancellation system that utilizes speakers 350 installed on a window shutter mechanism 352. FIG. 23 shows the characteristics of a panel, pipe, or flat speaker 350, and FIG. 24 shows the use of such speakers 350 for an open-air noise cancellation system for window or door applications. Since the sound produced by the panel, pipe, or flat speaker 350 travels as in the surface shape of the part of a cylinder, not as in the surface shape of the part of a sphere, there is little or no delay in sound travel between different target aligned positions horizontally (different positions 354 are depicted in FIG. 23, corresponding to T1-TN). This arrangement thus provides on time arrival of the canceling noise in a line making efficient noise cancellation.

FIG. 25 is a diagram that depicts a mechanical fin and blade assembly 360 for a noise cancellation system. Reference number 360a represents the assembly 360 in an open state, while reference number 360b represents the assembly 360 in a closed state. Assembly 360 includes frames 362, blades 364, and fins 366. These mechanical fins and extended blades are suitable for use with a practical embodiment. These elements are extended from the frame towards the noise source to create a longer path for the noise to travel, thus resulting in better absorption of noise by the absorptive materials applied to the frames. The blades 364 and the fins 366 may also be covered with absorptive materials. The fins 366 may be placed either downwards from the blade 364, upwards from the blade 364, or both. Whether the noise directly hits with an angle to the frame, or hits by diffractions because of the fins 366, the frames reduce the noise because the noise has a longer propagation path. The extended blades 364 and the fin 366 fit on top of each other as depicted in FIG. 25 when the shutter frames are closed. These blades 364 also help narrow the path the active noise cancellation speakers control, which increases the efficiency in canceling higher frequency noise even with a wider physical opening between the frames on the indoor side of the door/window.

The frames 362, blades 364, fins 366, and the cone of the speakers may be made of transparent or translucent materials such as polycarbonate and other plastic materials to increase the translucent or transparent quality of the window treatment. In such a translucent or transparent implementation, the shutter functions primarily as a noise reduction shutter rather than as a light blocking shutter. Of course, the level of transparency or opaqueness of the shutter can be adjusted to suit the needs of the particular application or location.

The system described herein allows cancellation and reduction of background noise such as the highway traffic noise, the airplane noise, industrial noise, air conditioner and home equipment noise, office noise, and other noise in the open-air environment, as an installed device. Systems, devices, and methods configured in accordance with various embodiments relate to:

A noise cancellation system for open-air applications. The system includes: open-air speakers configured for applying to open windows or door; a noise collection microphone located proximate to the open-air speakers, the noise-collection microphone being configured to obtain a noise signal; and a processor configured to generate a noise cancellation signal based upon the noise signal. The open-air system may be configured to have speaker box as rotating shutters. The open-air speakers may be configured to generate sound having a substantially cylindrical radiation pattern. The system may further comprise acoustic sound absorbing material located proximate to the open-air speakers. The system according may further comprise at least one acoustic blade and at least one fin located proximate to the open-air speakers.

A noise canceling window treatment comprising: a plurality of shutter vanes configured for mounting proximate to a window opening; open-air speakers configured for applying to a window or a door; at least one noise collection microphone coupled to the plurality of shutter vanes, the at least one noise collection microphone being configured to obtain a noise signal; a processor configured to generate a noise cancellation signal based upon the noise signal; and at least one speaker coupled to the plurality of shutter vanes, the at least one speaker being configured generate noise canceling sound based upon the noise signal. Each of the plurality of shutter vanes may include at least one noise collection microphone coupled thereto. Each of the plurality of shutter vanes may include at least one speaker coupled thereto.

Garden and Patio Umbrella Applications

An open-air noise cancellation system as described herein may also be suitably configured for use with an open garden or patio umbrella and other open air spaces. Such a system can reduce the effects of environmental noise by canceling noise in an open-air environment. The system provides effective open-air noise cancellation for open umbrella and other related applications. The example embodiment of the system has multiple microphones and speakers that are aligned, either paired or unpaired. Each microphone provides accurate information on the noise elements such as frequency, types, direction, and power of the environmental noises. Then, the noise information from the microphones is electronically processed to provide cancellation signals having opposite phase of the noise. The out-of-phase signals are transferred to amplifiers for output to the speakers for the same amount of sound but in opposite phases to cancel the original noises.

Various embodiments relate to the use of active noise cancellation techniques in a open garden umbrella and other applications suitable for use in an open-air environment. The system is utilized to reduce noise coming in beneath the open umbrella while people are sitting under the umbrella.

Conventional active noise cancellation techniques leverage the so-called “closed air” and “feed back” environment. Such techniques are commonly used in headsets and cellular phones. In contrast, however, a system configured as described in this section applies to the open-air environment, and such a system may employ one or more of the following techniques, features, and aspects (without limitation): active noise cancellation techniques; output power level control; frequency characteristic and control; acoustic elements to control sound and noise; and open air optimization to offset open air noise.

The system may also combine the use of flat and/or pipe speakers. In one practical embodiment, the system is realized as a umbrella which reduces the noise while people are seated below the system.

In one example embodiment, the system includes multiple sets of microphones and speakers. The microphones detect the noise, and the system processes the noise signals to create compensating or canceling electrical signals. The canceling signals are relayed to the speakers, which turn the signals back into sounds. The electronics create cancellation signals that are 180 degrees (within practical tolerances) out-of-phase with the actual noise signals. Thus, since the sounds from the speakers are of opposite phase from the noise, the generated sound actively cancels the unwanted noise sounds. The noise cancellation speakers add correcting sound waves that are simply out of phase with the unwanted noise, and provide significant reduction of background noise.

A system as described herein provides effective methods and apparatus for implementing open-air noise cancellation for open umbrella applications. The sound from the speakers is out-of-phase with the noise, thus canceling the noise sound. The noise cancellation speakers reproduce loud noises that are simply out-of-phase, thus performing significant reduction of background noise and producing a very quiet environment.

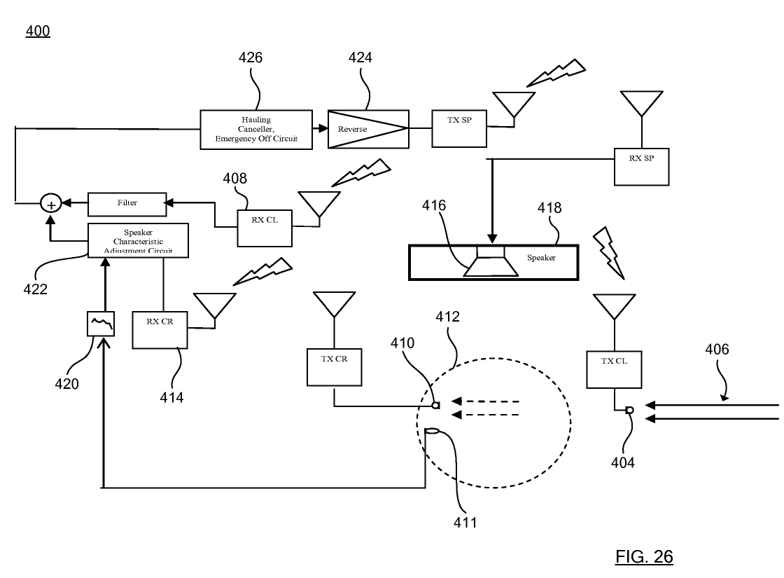

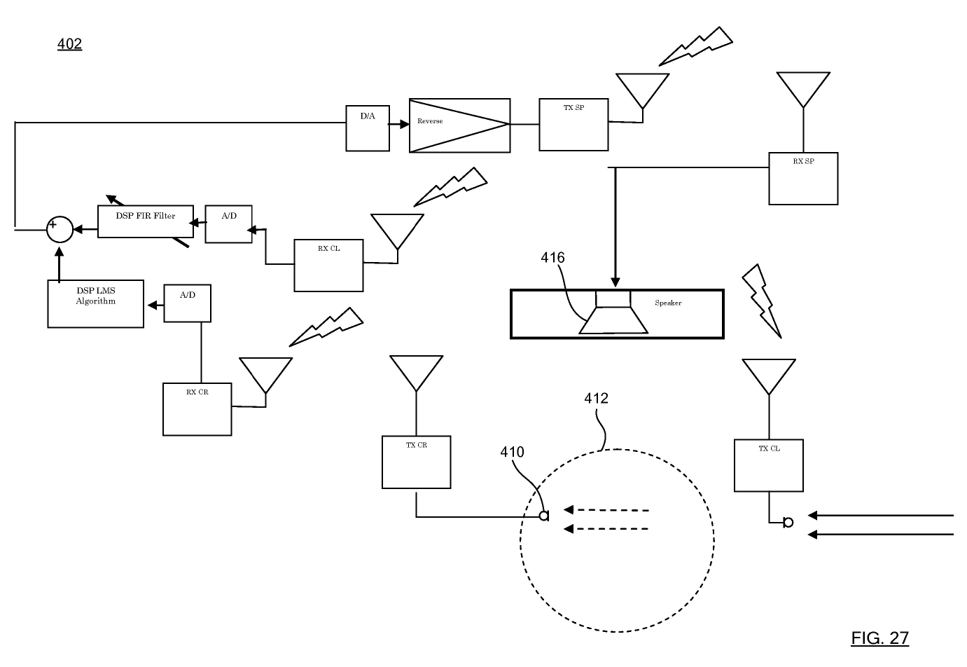

FIG. 26 is a schematic representation of an analog-based noise cancellation system 400 and FIG. 27 is a schematic representation of a digital-based noise cancellation system 402 suitable for use in an open-air environment. In both the analog system 400 and the digital system 402, the arrangement includes multiple sets of microphones and speakers.

Referring to analog system 400, at least one noise collection microphone 404 (also referred to as reference microphones) is strategically placed where the noise 406 is most apparent in the subject zone. The collected noise signal can be transmitted via a wireless or wired link to a suitable receiver point, for example, a receiver 408. The received signals can then be detected and processed. On the other hand, at least one error correction microphone 410/411 is strategically placed at the zone 412 where cancellation of noise is mostly targeted. This target zone 412 is preferably close to the human head, such as on the shoulder of a garden chair. The information collected by error correction microphone 410 can be transmitted via a wireless or wired communication technique to a suitable receiver point, for example, a receiver 414. In this example, at least one speaker 416 is placed above or beside the target noise cancellation zone 412. In both analog system 400 and digital system 402, the microphones detect the noise 406 approaching the target zone 412, then the detected signals are processed, with error corrections, and reversed to be 180 degrees out of phase relative to the noise 406. The correcting signals are then reproduced from the speakers 416, which may be connected using wireless and/or wired techniques and technologies. The active canceling sounds are produced from the speakers 416 to significantly reduce the noise in the targeted zone 412.

Analog system 400 may utilize components that were described above in the context of other applications and systems, and such components will not be redundantly described here in the context of analog system 400.

In the analog system 400, in order to effectively cancel the noise in target zone 412, it is important to measure the effect of sound travel (the distances from the noise collection microphone 404, the speaker 416, and the error correction microphone 410 to the target zone 412), sound frequency characteristics created by the microphone specifications, speaker specifications, and other acoustic impacts such as the form of the speaker box 418 and other objects. A sound characteristics acquisition system 420 essentially acquires such acoustic data at the time of development of the analog system 400. It measures the frequency characteristic and flatness of the sound system to properly reproduce opposite noise through the speakers 416. Once preset, the error correction microphone 410 collects the signals that are different from the preset signal characteristics, for purposes of adjustment in a speaker characteristic adjustment circuit 422. Then, proper levels of canceling noise dependent on such characteristics are reproduced and output through a reverse circuit 424 and the speaker 416. The hauling canceller and emergency shut off circuit 426 functions to reduce or shut off signals whenever there are excessive input to the microphones that would otherwise create abrupt loud sound though the system 400.

In digital system 402, a similar technique applies, however, in digital system 402, analog signals are converted to digital data and for digital processing by processing logic. Digital system 402 may share several components and respective functionality with analog system 400; common features and functionality will not be redundantly described in the context of digital system 402. In the digital system 402, a similar development method applies, however, in this system 402, analog signals are converted to digital data and processed digitally. An FIR filter and associated DSP estimate the acoustic characteristics from the sensor to the error correction microphone and generate the signals which are reversed and output through the speaker 416 for the target zone 412.

In connection with FIG. 26 and FIG. 27, the following acronyms may be used:

A/D: analog to digital signal converter;

D/A: digital to analog signal converter;

DSP: digital signal processing, which may be used to digitally process the signals;

FIR filter: finite impulse response filter, which may be used to estimate the acoustic response from the sensor to the error correction microphone 410;

LMS Algorithm: Least Mean Squares Algorithm, which may be used to generate error signals;

RX CL: Receiver, noise collection;

TX CL: Transmitter, noise collection;

RX CR: Receiver, error correction;

TX CR: Transmitter, error correction;

RX SP: Receiver, speaker sound; and

TX SP: Transmitter, speaker sound.

The number of microphones and speakers in a practical embodiment depends on the strategic noise canceling target zone size and other practical considerations.

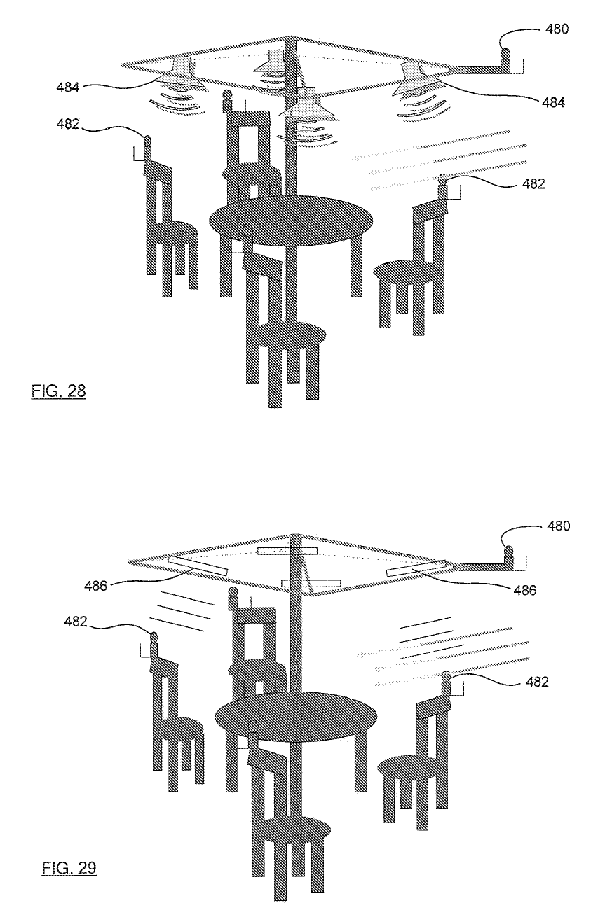

FIG. 28 is a diagram of one example embodiment of a noise cancellation system for an umbrella implementation. The microphones and the speakers are placed in strategic locations which may be wirelessly connected. FIG. 28 depicts one microphone 480 mounted to the umbrella itself, and a microphone 482 mounted to each chair. This example system also includes four speakers 484 mounted under the umbrella. In one practical embodiment, the speakers 484 are oriented to direct the sound toward the listeners seated under or near the umbrella.

FIG. 29 is a diagram of another example embodiment of a noise cancellation system for an umbrella implementation; this embodiment utilizes panel, pipe, or flat speakers 486 mounted to the umbrella. Each of the speakers 486 may have the characteristics described above in connection with FIG. 23. Since the sound produced by such a panel, pipe, or flat speaker 486 travels in the surface shape of a portion of a cylinder, not in the surface shape of a portion of a sphere, there is little or no delay in sound travel between the different target aligned positions horizontally (T1-TN), thus providing on time arrival of the canceling noise in a line and resulting in efficient noise cancellation.

The system described herein allows cancellation and reduction of background noise such as the highway traffic noise, the airplane noise, industrial noise, air conditioner and home equipment noise, office noise, and other noise in the open-air environment, as an installed device. Systems, devices, and methods configured in accordance with various embodiments relate to:

A noise cancellation system for open-air applications. The system comprises: open-air speakers configured for applying to open area such as under the garden umbrella; a noise collection microphone located proximate to the open-air speakers, the noise-collection microphone being configured to obtain a noise signal; and a processor configured to generate a noise cancellation signal based upon the noise signal. The system may be configured to have wireless or wired connections. The open-air speakers may be configured to generate sound having a substantially cylindrical radiation pattern.

A noise cancellation system for open-air applications. The system comprises: a covering for a local seating area; at least one chair for the local seating area; at least one noise collection microphone located proximate to the local seating area, the at least one noise collection microphone being configured to obtain a noise signal; a processor configured to generate a noise cancellation signal based upon the noise signal; and at least one speaker coupled to the covering, the at least one speaker being configured generate noise canceling sound based upon the noise signal. Each of the at least one chair may include at least one noise collection microphone coupled thereto. The covering may include at least one noise cancellation microphone coupled thereto. The covering may be an umbrella, an awning, or other architecture or arrangement.

Diffraction Control Applications

A system as described in this section is an open-air noise cancellation system suitable for diffraction control. A system is provided for reducing the effects of environmental noise by actively canceling noise which is diffracted over acoustic walls and other objects. The system reduces the amount of sound diffracted by the top edge of a wall, thus reducing the amount of environmental noise heard on the “protected” side of the wall. The example embodiment of the system has multiple microphones and speakers that are aligned, either paired or unpaired. Each microphone provides accurate information on the noise elements such as frequency, types, direction, and power of the environmental noises. Then, the noise information from the microphones is electronically processed to provide signals having the opposite phase of the noise signals. The out-of-phase signals are transferred to amplifiers for output to the speakers for the same amount of sound in opposite phases to cancel the original noise.

In general, when there are noise issues, sound walls are installed between the source and the receiver of the noise. The height, location, and the materials of the sound walls play a significant role in determining the effectiveness of the sound walls. In general, the closer the wall is placed to the sound source, or to the receiver, the better the noise reduction effect. The higher the wall, the better the effect would be for noise reduction. However, there are many height limitations in installing walls, and making the walls too high reduces brightness of the space and increases psychological pressures.

There are two practical measurement criteria in determining the materials and effectiveness of the sound walls. Transmission Loss (or Sound Transmission Class=STC) is the sound energy transmitted through the wall from the sound source to the receiver when the wall is installed on the line of sight point. Usually, high Transmission Loss of approximately 30 dBA is considered to be indicative of good sound barriers. Absorption Ratio (or Noise Reduction Co-efficiency=NRC) is the other criteria to determine how much of the sound energy is absorbed (and reflected) by such walls. For example, a wall with 0.90 rating means that 90% of the noise is absorbed, and 10% is reflected.

These criteria are good measurement criteria for the sound walls, however, there is another important path called the “diffraction path” where sound travels from the source to the receiver around an object. Diffraction is a physical phenomena where any waves, whether light, sound, or water travels around an object as if the waves bend around. In the case of a vertically deployed sound wall, sound bends at the top of the wall and travels towards the receive point of the sound. Other than direct sound paths, diffraction is one of the most significant paths of sound that can travel from the source to the receiver in an open air environment.

The diffraction amount depends on the length of the wave as well as the angles from the source through the object to the receiver. Sound with longer wavelength (lower frequency sound) diffracts more, and sound with shorter wavelength (higher frequency sound) diffracts less. The more angles from the source through the object to the receiver, the less sound is diffracted. Audible sound frequencies are between 20 Hz to 20,000 Hz, which corresponds to wavelengths between 17 mm up to 17 m. Sound travels at the speed of 340 meters per second, thus a frequency of 100 Hz corresponds to a 3.4 m wavelength, and a frequency of 1,000 Hz corresponds to a 34 cm wavelength.

The understanding of sound, reflection, absorption, and diffraction has been increased in the recent years and many improvements in sound walls have been implemented. However, traditional applications have not addressed diffraction patterns for purposes of diffraction control to effectively reduce the unwanted noise.

An apparatus or system as described herein provides effective methods of reducing the diffraction of environmental noise. In the practical embodiment, the mechanics and the electronics of a structure (which can be installed on the top side of a wall) includes microphones and speakers using active noise cancellation techniques. The system is utilized to reduce noise coming over the wall that may be diffracted towards the receiver. The system allows the walls to be lower while still achieving the same noise reduction effect as much higher walls.

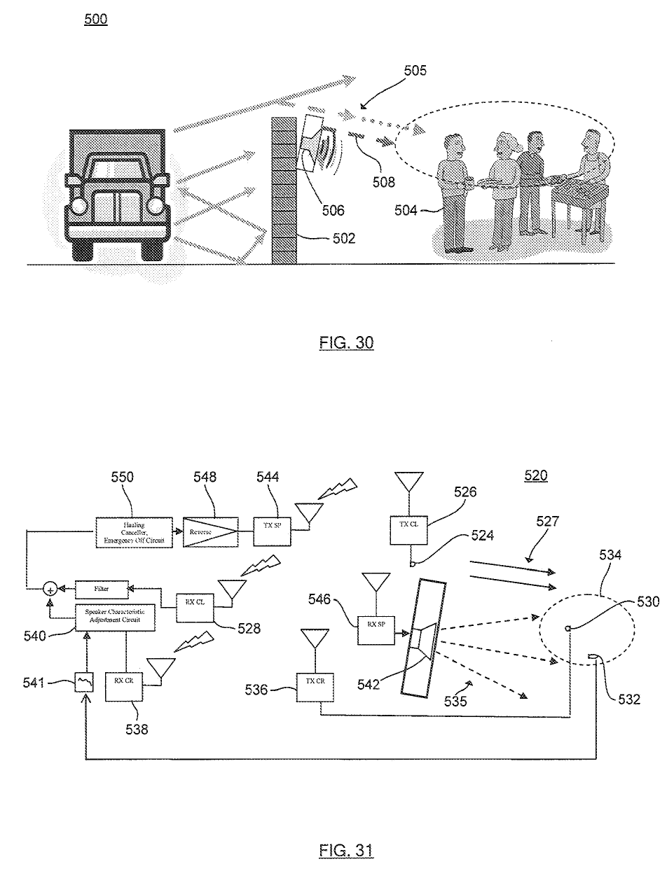

FIG. 30 is a diagram of an environment 500 having a noise cancellation system deployed therein. FIG. 30 illustrates how sound originating on one side of a wall 502 can travel over the wall 502, and how some of the sound is diffracted downward such that it travels to a receiver 504 located on the other side of the wall 502. In FIG. 30, the dashed arrow 505 represents this diffracted sound.

In this embodiment, the sound diffracts when it reaches the top of the wall 502, however, it is also cancelled by the mechanism and the electronic active noise canceling system (which is applied to the top side edge of the wall 502) as soon as the noise starts to travel around the edge towards the receiver 504. Briefly, environment 500 may include one or more noise cancellation speakers 506 that are mounted near the top edge of the wall 502. Speakers 506 can be angled downward such that canceling sound 508 is directed toward the receiver 504.

Conventional active noise cancellation techniques leverage the so-called “closed air” and “feed back” environment. Such techniques are commonly used in headsets and cellular phones. In contrast, however, an embodiment of this system applies to the open-air environment, and such a system may employ one or more of the following techniques, features, and aspects (without limitation): active noise cancellation techniques; output power level control; frequency characteristic and control; acoustic elements to control sound and noise; and open air optimization to offset open air noise.

The system may also combine the use of flat and/or pipe speakers as described above in the context of FIG. 12 and FIG. 23. In one practical embodiment, the system is realized in a speaker panel applied to the top side of the acoustic walls to have more efficient noise canceling effect.

In one example embodiment, the system includes multiple sets of microphones and speakers. The microphones detect the noise, generate electrical signals indicative of the detected noise, and relay them to the system processing core for suitable processing. The processing logic then generates noise cancellation signals to drive the speakers 506, which convert the noise cancellation signals into the canceling sound 508. The electronics of the system create cancellation signals that are 180 degrees (within practical tolerances) out-of-phase with the detected noise signals. Thus, since the sound from the speakers 506 are out-of-phase with the noise, the generated canceling sound 508 actively cancels the unwanted noise sounds. The noise cancellation speaker 506 adds corrective sound that is out of phase, and provides significant reduction of background noise.

A system as described herein provides effective methods and apparatus for implementing open-air noise cancellation for diffracted noise control. The sound from the speakers 506 is out-of-phase with the noise, thus canceling the noise sound. The noise cancellation speakers 506 reproduce sound that is out-of-phase with the unwanted noise, thus performing significant reduction of background noise and producing a very quiet environment.

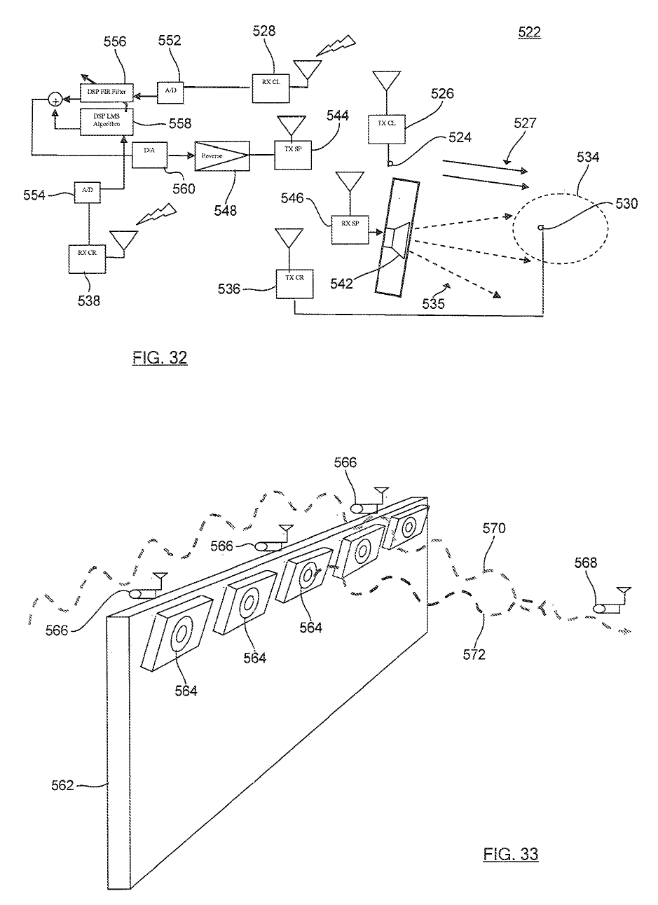

FIG. 31 is a schematic representation of an analog-based noise cancellation system 520 and FIG. 32 is a schematic representation of a digital-based noise cancellation system 522 suitable for use in, for example, environment 500. Both of these systems utilize multiple sets of microphones and speakers, even though only one speaker is depicted in FIG. 31 and FIG. 32. A number of the components of analog system 520 and digital system 522 were described above in the context of other embodiments. For the sake of brevity, such common features and functionality will not be redundantly described in detail in the context of analog system 520 and digital system 522.

Analog system 520 includes at least one noise collection microphone 524 (also referred to herein as reference microphones) that configured for placement along a wall towards the noise source side where the noise is most apparent. Microphone 524 may include or be coupled to a suitably configured transmitter 526 that facilitates transmission (via a wireless and/or a wired data communication link) of data indicative of the detected noise, which may include diffracted noise 527. In this example, transmitter 526 communicates with a receiver 528 associated with the processing architecture of the system. The processing architecture, which may include any number of cooperating elements, components, or subsystems, detects and processes the received signals in the manner described herein.

Analog system 520 may also include at least one error correction microphone 530/532, which can be strategically placed in or near the target zone 534 where cancellation of noise is desired, such as the side of the wall opposite to the noise source, along the line where the diffracted noise 527 and canceling noise 535 mix, in a desired quiet zone, or the like. Error correction microphone 530 may include or be coupled to a transmitter 536 that facilitates transmission (via a wireless and/or a wired data communication link) of data indicative of the detected sound. In this example, transmitter 536 is configured to transmit information to a receiver 538 associated with the processing architecture of the system. More specifically, receiver 538 may be coupled to a speaker characteristic adjustment circuit 540 (described below). Error correction microphone 532 may also communicate (wirelessly and/or via a wired link) with processing architecture. Here, error correction microphone 532 sends detected sound signals to a sound characteristics acquisition system 541 (described below).

Analog system 520 includes one or more speakers 542 that are suitably configured for placement on the top side of the wall. In both analog system 520 and digital system 522, the microphones detect the noise approaching the wall or coming over the wall, then the detected signals are processed, with error corrections, and the processed signals are reversed by 180 degrees before being reproduced from the speakers 542. The speakers 542 may be connected to the processing architecture using a wireless link and/or a wired link. In this embodiment, the processing architecture includes or communicates with a transmitter 544 that sends noise canceling signals to a receiver 546 associated with speaker 542. The signals received by receiver 546 are then used to drive speaker 542. In this manner, the active canceling sounds are produced from the speakers 542, thus canceling the diffracted noise, and significantly reducing the noise in the targeted zone 534.

In analog system 520, in order to effectively cancel noise in target zone 534, it is important to measure the delay effect of sound travel (spanning the distances from the noise collection microphone 524, the speaker 542, and the error correction microphones 530/532 to the target zone 534). It may also be desirable to measure sound frequency characteristics created by the microphone specifications, speaker specifications, and other acoustic impacts such as the form of the enclosure for speaker 542 and other objects. The sound characteristics acquisition system 541 essentially acquires such acoustic data at the time of development of the analog system 520. It measures the frequency characteristic and flatness of the sound system to properly reproduce out-of-phase signals through the speakers 542. Once preset, the error correction microphone 532 collects the signals that are different from the preset signal characteristics, and sound characteristics acquisition system 541 can initiate suitable adjustments in speaker characteristic adjustment circuit 540. Then, appropriate levels of canceling noise, which may be dependent on such characteristics, are reproduced and output through a reverse circuit 548 and the speaker 542. A hauling canceller and emergency shut of circuit 550 functions to reduce or shut off signals whenever there are excessive inputs to the microphones that might otherwise create abrupt loud sound though the analog system 520.

Digital system 522 may include components described above in the context of analog system 520, for example: noise collection microphone 524; transmitter 526; receiver 528; error correction microphone 530; speaker(s) 542; transmitter 544; receiver 546; transmitter 536; receiver 538; and reverse circuit 548. These items will not be redundantly described in the context of digital system 522.

In digital system 522, analog signals are converted to digital data to facilitate digital processing by the processing architecture. For example, the analog signals received by receiver 528 may be converted into corresponding digital representations by an analog-to-digital converter 552, and the analog signals received by receiver 538 may be converted into corresponding digital representations by an analog-to-digital converter 554. The digital output of analog-to-digital converter 552 is provided to an FIR filter 556, and the digital output of analog-to-digital converter 554 is provided to an LMS algorithm module 558. As depicted in FIG. 32, FIR filter 556 may be actively adjusted by LMS algorithm module 558. FIR filter 556 is suitably configured to estimate the acoustic characteristics from the microphone 524 to the error correction microphone 530, and to generate a digital output. This digital output is summed with the output of LMS algorithm module 558, and converted into an analog signal by a digital-to-analog converter 560. The analog output of digital-to-analog converter 560 serves as an input to reverse circuit 548, which reverses this signal and provides it to transmitter 544 for communication to speaker 542. The signal received by receiver 546 represents the drive signal for speaker 542.

In FIGS. 31 and 32, the following acronyms may be used:

A/D: analog to digital signal converter;

D/A: digital to analog signal converter;

DSP: digital signal processing, which may be used to digitally process the signals;

FIR filter: finite impulse response filter, which may be used to estimate the acoustic response from the microphone 524 to the error correction microphone 530;

LMS Algorithm: Least Mean Squares Algorithm, which may be used to generate error signals;

RX CL: Receiver, noise collection;

TX CL: Transmitter, noise collection;

RX CR: Receiver, error correction;

TX CR: Transmitter, error correction;

RX SP: Receiver, speaker sound; and

TX SP: Transmitter, speaker sound.

In the practical embodiment, the number of microphones and the number of speakers that are aligned along the top side of the wall depends on the length of the wall and the size of the target area 534. The intervals between the speakers are preferably less than 250 cm to effectively cancel open air noise in this application. Closer spacing between speakers is usually better to cancel higher frequency range signals. For example, 250 cm is equal to approximately half wavelength of 680 Hz, thus making the noise cancellation effective below that frequency.

FIG. 33 is a perspective view of a wall 562 having noise cancellation speakers 564 mounted thereon. In this embodiment, the noise cancellation system employs five speakers 564, although the specific number of speakers 564 may vary depending upon the system environment, the size of the wall, the size of the target zone, etc. One or more reference or noise collection microphones 566 are located on the noisy side of the wall 562. In this embodiment, the system uses three noise collection microphones 566 in a cooperative manner. The speakers 564 emit correcting sound waves toward the target zone, and one or more error correction microphones 568 are placed in strategic places in or near the target zone. In FIG. 33, the diffracted noise 570 is shown traveling over the wall 562, and the canceling signal 572 is shown being generated from the speakers 564 mounted to the “quiet” side of the wall 562.

Although not a requirement in all embodiments, the system shown in FIG. 33 employs noise collection microphones 566 and error correction microphone(s) 568 having wireless data communication capability. Such wireless connectivity enables flexible and clean installation of these microphones in the environment. As described above in connection with FIG. 31 and FIG. 32, wireless signals can be transmitted from these microphones to the processing architecture utilized by the noise cancellation system.

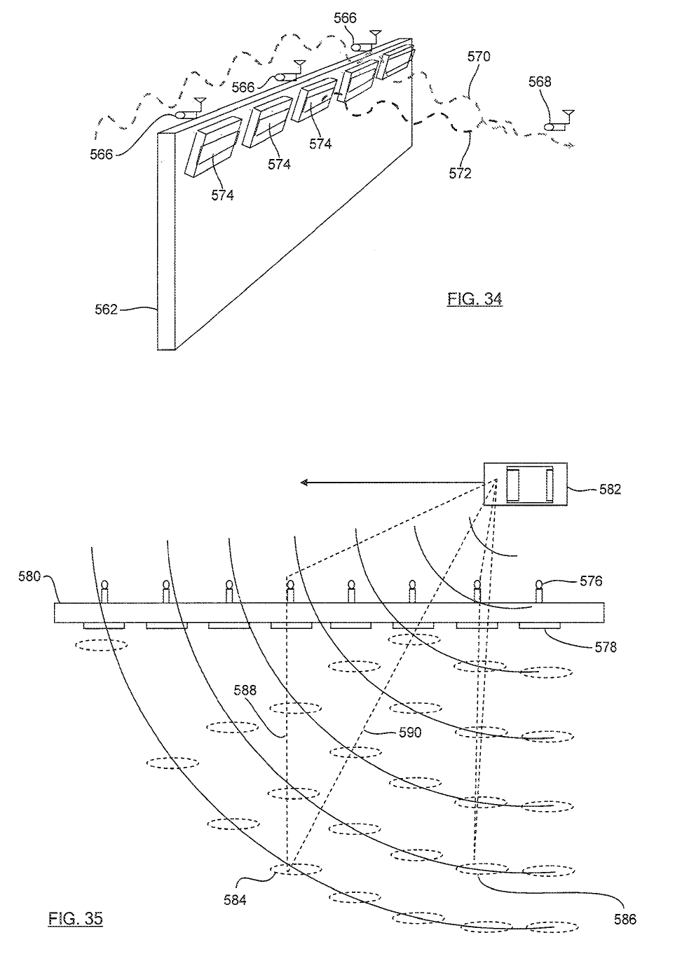

FIG. 34 is a perspective view of another wall 562 having noise cancellation speakers 574 mounted thereon. The system shown in FIG. 34 is similar to the system shown in FIG. 32, however, the system shown in FIG. 34 utilizes panel, pipe, or flat speakers 574 rather than round/cone speakers. One example of such a speaker 574 is depicted in FIG. 23 (see the above description of speaker 350). When used in connection with a sound diffraction control application, the sound produced by the panel, pipe, or flat speaker 574 has traveling shape that resembles a portion of a cylinder, in contrast to a shape that resembles a portion of a sphere (which may result from a point source speaker or a round speaker). Therefore, there is little or no delay in sound waves corresponding to different horizontal positions relative to the long dimension of the speaker 574, and speaker 574 provides on time arrival of the canceling noise in a line, which results in efficient and effective noise cancellation.

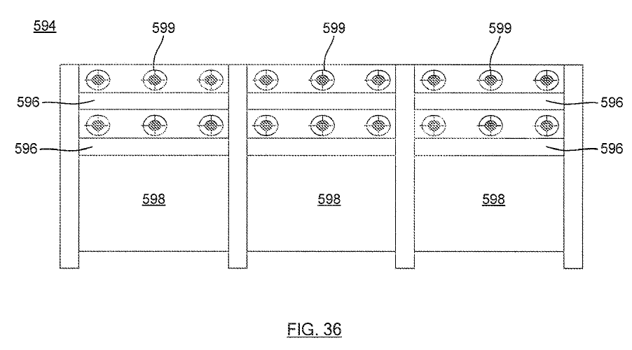

FIG. 35 is a diagrammatic top view of a moving target detection and adjustment system. Moving target detection and adjustment can be utilized in connection with the systems shown in FIG. 33 and FIG. 34. FIG. 35 shows aligned sets of noise collection microphones 576 and noise cancellation speakers 578 coupled to a wall 580. FIG. 35 depicts a moving sound source 582, which may be a car, a motorcycle, or the like. Since in many cases the sound sources are moving (for example, aircraft, automobiles, or the like), each microphone 576 detects the sound at each position, then sends information to noise cancellation units that correspond to each microphone-speaker set. Appropriate characteristics of sound at the time the sound reaches each respective microphone 576 are processed in the noise cancellation units. These characteristics may include, without limitation, the timing or the phase of the sound waves (or sound pressures) for each frequency, for example, a frequency centered at 250 Hz, a frequency centered at 500 Hz, and so on. Because the timing associated with the sound from the noise source 582 reaching the various microphones 576 is different, the phases of sound are different at the time they reach each microphone 576. For example, when a 250 Hz centered frequency sound is processed, because the full wavelength is approximately 136 centimeters, the phases that such sound arrives at each microphone 576 are different. The noise cancellation units continuously monitor the differences. The noise cancellation system will then process the detected sounds to reproduce the out-of-phase sounds from the speakers 578 to most efficiently cancel the noise. In other words, in order to reach the listening point 584, one speaker 578 may be producing an out of phase signal at one particular delayed phase and one loudness associated to it, and another speaker 578 may be producing an out of phase signal at another slightly different and delayed phase and another loudness, so that in aggregate, they match the appropriate opposite phase and the loudness of the original noise 582 that reaches the listening point 584.

The signal levels obtained from two or more microphones 576 for a particular frequency are compared at the signal processing electronics level, which also calculates the phase differences and intentionally delays the reproduction of the sound wave from the speakers 578 to match the next or any matching phase of the wave to compensate for the distance and speed of the sound source reaching the target zone. The signal levels are obtained for different sampled frequencies, where the sampling frequencies can be adjusted up to, for example, 48 kHz. This obtaining step can be repeated for different frequencies and the so-called step size can be adjusted. For example, at the moment of time frozen in FIG. 35, the system processes multiple frequencies for each of the different microphone signals. The phase differences calculated here relate to the phase difference of the sound detected from one microphone to another; since all systems are active, they continually adjust. The phase differences are calculated using multichannel processing from one system to another; one noise cancelling unit is compared to another noise cancelling unit. The delay associated with the speakers may be adjustable. It could be a few speakers or many. The adjustment and delay depends on the target noise reduction level, the distance from the noise source and, thus, the phase difference between the microphone positions, etc. The further the noise source, the less phase difference. In practice, the system may not actually “predict” the next sound wave; however, many noise sources are repeatable and somewhat predictable (e.g., engine noise) such that, as soon as they are detected, the system can do the adaptation, comparison, and adjustment very quickly. In FIG. 35, a number of small target zones are depicted as dashed ovals. There is very small phase delay at the target zone 586 between the sound traveling directly from the sound source 582 versus the sound obtained through the second microphone (relative to the right side of FIG. 35) and reproduced by the second speaker except for the electronic delay inside the processors which is minimal compared to the delay of the sound travel. At the 584 target zone, however, since the canceling noise generally travels along the line 588 to reach to the microphone, reversed to the opposite phase, and reproduced by the fifth speaker, such travel delay is adjusted. For example, when the sound reaches the third microphone from the right, it is immediately processed by the noise cancellation unit. There is very little delay between the original sound path to get to point 584 and the out of phase cancelling noise generated by the third speaker to get to point 586, so the loudness and the phase do not have to be adjusted. The line 590 represents this sound path. However, when the sound reaches the fifth microphone, the phase of the sound is different; it is delayed. The direct path to point 584 is shorter, but the overall distance from the source 582, to the fifth microphone, then to the target 584 along line 588 is longer. The system adjusts the delay to the next phase to reach to the target. The phase differences between the microphones aligned with the points 584 and 586 are collected and the phase differences are adjusted to the pre-set delay functions. This process is continuously performed for different possible timing and for the range of sound characteristics of the noise source and targeted to be cancelled so that the canceling effects are most efficiently performed. Since each microphone is detecting the noise at that point, the system also adjusts the frequency change caused by the so called Doppler Effect for the high speed moving sound source.

FIG. 36 is a front view of an example wall-mounted noise diffraction control system. This open air control system can also be utilized for walls and gates with many open spaces or holes formed therein. In this example, a wall structure 594 includes open space 596 between acoustic material, and sound passing through the open space 596 can be controlled by the noise cancellation system. Transparent acoustic materials 598 with certain thickness can be used for see-through and air-through applications. Such acoustic materials may also be utilized to mount noise cancellation speakers 599. FIG. 36 illustrates one embodiment where two rows of speakers 599 are mounted to slats of the wall structure 594, where open space 596 divides the slats.

The system may also be used in combination with certain top edge structures (as described above in connection with FIGS. 2-6) that employ porous absorptive material configured to further reduce the amount of sound wave diffraction over the top of the wall structure 594. The acoustic structure determines the diffraction amount and thus defines the level of control by the noise cancel system.

The system described herein allows cancellation and reduction of background noise such as the highway traffic noise, the airplane noise, industrial noise, air conditioner and home equipment noise, office noise, and other noise in the open-air environment, as an installed device.

While at least one example embodiment has been presented in the foregoing detailed description, it should be appreciated that a vast number of variations exist. It should also be appreciated that the example embodiment or embodiments described herein are not intended to limit the scope, applicability, or configuration of the invention in any way. Rather, the foregoing detailed description will provide those skilled in the art with a convenient road map for implementing the described embodiment or embodiments. It should be understood that various changes can be made in the function and arrangement of elements without departing from the scope of the invention.

What is claimed is:

1. A noise cancellation system comprising:
an open-air speaker configured to be mounted to a wall;
a noise collection microphone located proximate to the open-air speaker, the noise-collection microphone being configured to obtain a noise signal; and
a processing architecture coupled to the open-air speaker and coupled to the noise collection microphone, the processing architecture being configured to generate a noise cancellation signal based upon the noise signal.

2. A system according to claim 1, the open-air speaker being coupled to the processing architecture via a wireless link.

3. A system according to claim 1, the noise collection microphone being coupled to the processing architecture via a wireless link.

4. A system according to claim 1, the open-air speaker being configured to generate sound having a substantially cylindrical radiation pattern.

5. A system according to claim 1, wherein:
the noise signal is generated by a moving noise source; and
the processing architecture is configured to generate the noise cancellation signal in response to motion of the noise source.

6. A system according to claim 1, the processing architecture being configured to generate the noise cancellation signal in response to frequencies of the noise signal.

7. A system according to claim 1, the processing architecture being configured to generate the noise cancellation signal in response to levels of the noise signal.

8. A system according to claim 1, further comprising acoustic sound absorbing material located proximate to said open-air speaker.

9. A system according to claim 1, further comprising a diffraction control mechanism coupled to the wall, the diffraction control mechanism being configured to reduce diffraction of the noise signal over the wall.

10. A system according to claim 1, the processing architecture comprising a speaker characteristic adjustment circuit configured to influence the noise cancellation signal in response to acoustic characteristics of the system.

11. A system according to claim 1, the processing architecture comprising a sound characteristics acquisition system configured to acquire acoustic characteristics of the open-air speaker and the noise collection microphone.

12. A system according to claim 1, further comprising:
at least one additional open-air speaker configured to be mounted to the wall; and
at least one additional noise collection microphone; wherein
the open-air speakers and the noise collection microphones mounted to the wall in a paired alignment; and
the processing architecture is configured to perform moving target detection and adjustment in response to phase delay of the noise signal relative to the open-air speakers and the noise collection microphones.

13. A noise cancellation system comprising:
a sound barrier wall having a noise source side and a quiet side;
at least one noise collection microphone located on the noise source side, and being configured to obtain a noise signal;
at least one open-air speaker located on the quiet side, and being configured to generate a noise cancellation signal;
at least one error correction microphone located on the quiet side, and being configured to obtain an error correction signal; and
a processing architecture configured to generate a noise cancellation signal based upon the noise signal and based upon the error correction signal.

14. A noise cancellation system according to claim 13, wherein:
the sound barrier wall comprises slats that define open air spaces; and
the at least one open-air speaker is mounted to the slats.

15. A noise cancellation system according to claim 14, the slats being formed from acoustic material.

16. A noise cancellation system comprising:
a sound barrier wall having a noisy side and a quiet side;
a plurality of noise collection microphones mounted proximate the top of the sound barrier wall in a spaced pattern, the plurality of noise collection microphones being configured to collect noise signals;
a plurality of noise cancellation speakers mounted to the quiet side of the sound barrier wall in a spaced pattern and aligned with the plurality of noise collection microphones, the plurality of noise cancellation speakers being configured to generate noise cancellation signals; and
a processing architecture configured to:
process the noise signals;
generate noise cancellation signals in response to the noise signals and in response to phase delays associated with the plurality of noise collection microphones and the plurality of noise cancellation speakers; and
drive the plurality of noise cancellation speakers with the noise cancellation signals.

17. A system according to claim 16, each of the plurality of noise cancellation speakers being configured to generate sound having a substantially cylindrical radiation pattern.

18. A system according to claim 16, wherein:
the noise signals are generated by a moving noise source; and
the processing architecture is configured to generate the noise cancellation signals in response to motion of the noise source.

19. A system according to claim 16, further comprising acoustic sound absorbing material located proximate the top of the wall.

20. A system according to claim 16, further comprising a diffraction control mechanism coupled to the wall, the diffraction control mechanism being configured to reduce diffraction of the noise signals over the wall.
